(19) United States
(12) Patent Application Publication
(10) Pub. No.: US 2011/0217280 A1
(43) Pub. Date: Sep. 8, 2011

(54) METHODS FOR TREATING NEUROPSYCHIATRIC CONDITIONS

(76) Inventors: Benedikt Vollrath, San Diego, CA (US); Jay Lichter, Rancho Santa Fe, CA (US)

(21) Appl. No.: 12/809,038

(22) PCT Filed: Dec. 19, 2008
(86) PCT No.: PCT/US08/87834

§ 371 Date: May 11, 2011

Related U.S. Application Data
(60) Provisional application No. 61/015,145, filed on Dec. 19, 2007.

(51) Int. Cl. A61K 38/45; A61K 38/18; A61K 38/46; A61K 31/53; A61P 25/00; A61P 25/18; A61P 25/24

(52) U.S. Cl. 424/94.5; 514/8.3; 424/94.6; 514/241

(57) ABSTRACT

Provided herein are methods for treating a subject suffering from a neuropsychiatric condition (e.g., schizophrenia). The methods include systemic administration of a pharmacological composition containing a therapeutically effective amount of a PAK activator.

### CROSS REFERENCE

This application claims the benefit of U.S. provisional application Ser. No. 61/015,145 filed Dec. 19, 2007, which is incorporated by reference in its entirety.

### BACKGROUND OF THE INVENTION

Neuropsychiatric conditions (NCs) are characterized by a variety of debilitating affective and cognitive impairments. For example, in schizophrenia, one of the most common psychotic disorders, individuals may suffer from hallucinations, disorders of movement, and the inability to initiate plans, speak, or express emotion. Cognitive deficits in schizophrenia include problems with attention, memory, and the executive functions that allow us to plan and organize. Other NCs include, e.g., mood disorders, age-related cognitive decline, and neurological disorders (e.g., epilepsy and huntington's disease). The effects of NCs are devastating to the quality of life of those afflicted as well as that of their families. Moreover, NCs impose an enormous health care burden on society. A number of NCs have been associated with alterations in the morphology and/or density of dendritic spines, membranous protrusions from dendritic shafts of neurons that serve as highly specialized structures for the formation, maintenance, and function of synapses.

### SUMMARY OF THE INVENTION

Described herein are methods and compositions for treating a subject suffering from a neuropsychiatric condition (e.g., schizophrenia, clinical depression, age-related cognitive decline, and epilepsy) by administering to the subject a pharmaceutical composition comprising a therapeutically effective amount of an activator of a p21-activated kinase (PAK), e.g., PAK1, PAK2 or PAK3, as described herein. PAK activation is shown to play a key role in spine morphogenesis, and activators of PAK are administered to drive an increase in spine morphogenesis and/or rescue defects in subjects suffering from a condition in which dendritic spine morphology, density, size, motility, plasticity and/or function are aberrant, including but not limited to lower than normal spine density, a reduction in spine size, defective spine morphology, a reduction in spine plasticity, or a reduction in spine motility.

Accordingly in one aspect provided herein is a method for treating a subject suffering from a neuropsychiatric condition, comprising administering to the subject a pharmacological composition comprising a therapeutically effective amount of at least one activator of a p21-activated kinase, wherein the neuropsychiatric condition is associated with abnormal (e.g., lower than normal) dendritic spine density, a reduction in spine size, a reduction in spine plasticity, or a reduction in spine motility. In some embodiments, the neuropsychiatric condition is a psychotic, cognitive, or mood disorder. In some embodiments, the neuropsychiatric condition is associated with abnormal (e.g., lower than normal) spine density. In some embodiments, the neuropsychiatric condition is a psychotic disorder.

In some embodiments, the neuropsychiatric condition is schizophrenia, clinical depression, epilepsy, age-related cognitive decline, Huntington's disease, Down's syndrome, Niemann-Pick disease, spongiform encephalitis, Lafora disease, Maple syrup urine disease, maternal phenylketonuria, atypical phenylketonuria, or tuberous sclerosis. In some embodiments, the neuropsychiatric condition is schizophrenia. In some embodiments, the subject suffering from schizophrenia is administered, in addition to a PAK activator composition, a therapeutically effective amount of an antipsychotic drug.

In some embodiments, the neuropsychiatric condition to be treated is clinical depression. In some embodiments, the subject suffering from clinical depression is administered, in addition to a PAK activator composition, a therapeutically effective amount of an antidepressant drug.

In some embodiments, the pharmacological composition to be administered contains at least one indirect PAK activator. In some embodiments, an indirect PAK activator is a TrkB receptor agonist, an inhibitor of FMRP binding to p21-activated kinase, an inhibitor to FMRP binding to p21-activated kinase mRNA, an inhibitor of FMRP expression, an activator of p21 kinase, an activator of Rac, an activator of Cdc42, an activator of NCK, and activator of GRB2, an activator of PDK1, an inhibitor of CDK5, an activator of a PI3 kinase or any combination thereof. In some embodiments, the inhibitor of FMRP expression comprises an FMRP RNAi, an FMRP antisense nucleic acid, an FMRP ribozyme, or any combination thereof. In some embodiments, the TrkB receptor agonist is a small molecule agonist. In some embodiments, the TrkB receptor agonist is a blood-brain barrier-permeable form of BDNF.

In some embodiments a PAK activator is a direct activator. In some embodiments, the direct activator comprises a constitutively active form of p21 kinase, Rac, or Cdc42.

In some embodiments, the at least one activator is an activator of PAK1. In some embodiments, the at least one activator is an activator of PAK2. In some embodiments, the at least one activator is an activator of PAK3.

In a related aspect provided herein is a method for treating a subject suffering from schizophrenia, comprising administering to the subject a pharmacological composition comprising a therapeutically effective amount of an activator of a p21-activated kinase.

In another aspect provided herein is a method for treating a subject suffering from a mood disorder, comprising administering to the subject a pharmacological composition comprising a therapeutically effective amount of an activator of a p21-activated kinase. In some embodiments, the mood disorder is clinical depression.

In a further provided herein is a method for treating a subject suffering from Huntington's disease, comprising administering to the subject a pharmacological composition comprising a therapeutically effective amount of an activator of a p21-activated kinase.

In yet another aspect provided herein is a method for treating a subject suffering from age-related cognitive decline, comprising administering to the subject a pharmacological composition comprising a therapeutically effective amount of an activator of a p21-activated kinase.

In another aspect provided herein is a method for treating a subject suffering from epilepsy, comprising administering to the subject a pharmacological composition comprising a therapeutically effective amount of an activator of a p21 activated kinase.

In one aspect provided herein is a method for reversing some or all defects in dendritic spine morphology, spine size and/or spine plasticity in a subject with a neuropsychiatric condition or predicted to develop a neuropsychiatric condition, comprising administering to the subject a pharmacological composition comprising a therapeutically effective amount of a PAK activator. I PAK antagonist. The subject is in some preferred embodiments a human.

### CERTAIN DEFINITIONS

As used herein the term “Treatment” or “treating” includes achieving a therapeutic benefit and/or a prophylactic benefit. By therapeutic benefit is meant eradication or amelioration of the underlying disorder or condition being treated. For example, in an individual with schizophrenia, therapeutic benefit includes partial or complete halting of the progression of the disorder, or partial or complete reversal of the disorder. Also, a therapeutic benefit is achieved with the eradication or amelioration of one or more of the physiological or psychological symptoms associated with the underlying condition such that an improvement is observed in the patient, notwithstanding the fact that the patient is still affected by the condition. A prophylactic benefit of treatment includes prevention of a condition, retarding the progress of a condition, or decreasing the likelihood of occurrence of a condition. As used herein, “treating” or “treatment” includes prophylaxis.

As used herein, the phrase “neuropsychiatric condition” refers to any condition, other than Alzheimer's Disease or Fragile-X Mental Retardation, that results in chronic impairment in cognition, affect, or motor function.

As used herein, the phrase “psychotic disorder” refers to a severe mental disorder characterized by derangement of personality and loss of contact with reality and causing deterioration of normal social functioning. Examples of psychotic disorders include, but are not limited to, schizophrenia, schizoaffective disorder, schizophreniform disorder, brief psychotic disorder, delusional disorder, shared psychotic disorder (Folie a Deux), substance induced psychosis, and psychosis due to a general medical condition.

As used herein, the phrase “cognitive disorder”) refers to any chronic condition, other than Alzheimer's disease, that impairs reasoning ability, e.g., age-related cognitive decline.

As used herein, the phrase “reduction in spine size” refers to decreased dendritic spine volumes or dendritic spine surface areas associated with a neuropsychiatric condition relative to spine volumes or surface areas in the same brain region (e.g., the CA1 region, prefrontal cortex) in a normal subject (e.g., a mouse, rat, or human) of the same age. The phrase “defective spine morphology” refers to abnormal dendritic spine shapes associated with a neuropsychiatric condition relative to the dendritic spine shapes in the same region in a normal subject (e.g., a mouse, rat, or human) of the same age. The phrase “reduction in spine plasticity” refers to an impairment in the ability of dendritic spines to undergo stimulus-dependent morphological or functional synaptic or post-synaptic changes (e.g., calcium entry through NMDA receptors, LTP, LTD, etc) associated with a neuropsychiatric condition as compared to dendritic spines in the same brain region in a normal subject of the same age. The phrase “reduction in spine motility” refers to an impairment in the ability of dendritic spines to move in response to synaptic or pharmacological stimuli (e.g., actin-based movement) associated with a neuropsychiatric condition as compared to dendritic spines in the same brain region in a normal subject of the same age.

As used herein, the term “agonist” refers to a molecule which is capable of activating one or more of the biological activities of a target molecule, such as a TrkB receptor, an Eph receptor, or an NMDA receptor. Agonists or activators, for example, act by activating a target molecule and/or mediating signal transduction. In some embodiments, the phrase “partial agonist” or “partial activator” refers to a molecule which can induce a partial response. In some instances, a partial agonist or partial activator mimics the spatial arrangement, electronic properties, or some other physicochemical and/or biological property of the agonist or activator. In some instances, in the presence of elevated levels of an agonist or an activator, a partial activator or a partial agonist competes with the agonist or activator for occupancy of the target molecule and provides a reduction in efficacy, relative to the agonist or activator alone. For target molecules that are constitutively biologically active, the phrase “inverse agonist” refers to a molecule that reverses the constitutive biological activity of a target molecule. In some embodiments, a PAK activator described herein is a partial activator of a PAK.

As used herein, the phrase “biologically active” refers to a characteristic of any substance that has activity in a biological system and/or organism. For instance, a substance that, when administered to an organism, has a biological effect on that organism, is considered to be biologically active. In particular embodiments, where a protein or polypeptide is biologically active, a portion of that protein or polypeptide that shares at least one biological activity of the protein or polypeptide is typically referred to as a “biologically active” portion.

As used herein, the term “effective amount” is an amount, which when administered systemically, is sufficient to effect beneficial or desired results, such as beneficial or desired clinical results, or enhanced cognition, memory, mood, or other desired effects. An effective amount is also an amount that produces a prophylactic effect, e.g., an amount that delays, reduces, or eliminates the appearance of a pathological or undesired condition. Such conditions include, but are not limited to, schizophrenia, clinical depression, epilepsy, age-related cognitive decline, Huntington's disease, Down's syndrome, Niemann Pick disease, spongiform encephalitis, Lafora disease, Maple syrup urine disease, maternal phenylketonuria, atypical phenylketonuria, or tuberous sclerosis. An effective amount is optionally administered in one or more administrations. In terms of treatment, an “effective amount” of a composition described herein is an amount that is sufficient to palliate, ameliorate, stabilize, reverse or slow the progression of an NC, e.g., age-related cognitive decline. An “effective amount” includes any PAK activator used alone or in conjunction with one or more agents used to treat a disease or disorder. An “effective amount” of a therapeutic agent as described herein will be determined by a patient's attending physician or other medical care provider. Factors which influence what a therapeutically effective amount will be include, the absorption profile (e.g., its rate of uptake into the brain) of a PAK activator, time elapsed since the initiation of the NC, and the age, physical condition, existence of other disease states, and nutritional status of the individual being treated. Additionally, other medication the patient is receiving, e.g., antipsychotic drugs used in combination with a PAK activator, will typically affect the determination of the therapeutically effective amount of the therapeutic agent to be administered.

As used herein, the phrase “an agent that facilitates the transport of the PAK activator across the blood brain bather” refers to an agent that mediates, facilitates and/or enhances penetration of a compound described herein through the blood brain barrier. In some embodiments, a blood brain barrier facilitator increases influx of a compound described herein. In some instances, an increase in influx of a compound described herein across the blood brain barrier is achieved by modulating the lipophilic nature of a compound described herein (e.g., via conjugation of a low density lipid particle to a compound described herein). In some instances, an increase in influx of a compound described herein across the blood brain barrier is achieved by modifying a compound described herein (e.g., by reducing or increasing the number of charged groups on the compound) and enhancing affinity for a blood brain barrier transporter. In some embodiments, a blood brain barrier facilitator reduces or inhibits the efflux of a compound described herein from the blood brain barrier (e.g., an agent that suppresses P-glycoprotein pump mediated efflux).

As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end formation); (3) translation of an RNA into a polypeptide or protein; (4) post-translational modification of a polypeptide or protein.

As used herein the term “PAK polypeptide” or “PAK protein” refers to a protein that belongs in the family of p21-activated serine/threonine protein kinases. These include mammalian isoform identified, e.g., PAK1, PAK2, PAK3, PAK-4, PAK5, and/or PAK6; and/or lower eukaryotic isoforms, such as the yeast Ste20 (Leberter et al., 1992, EMBO J., 11:4805; incorporated herein by reference) and/or the Dictyostelium single-headed myosin I heavy chain kinases (Wu et al., 1996, J. Biol. Chem., 271:31787; incorporated herein by reference). Representative examples of PAK include, but are not limited to, human PAK1 (GenBank Accession Number AAA65441), human PAK2 (GenBank Accession Number AAA65442), human PAK3 (GenBank Accession Number AAC36097), human PAK 4 (GenBank Accession Numbers NP_{—}005875 and CAA09820), human PAK5 (GenBank Accession Numbers CAC18720 and BAA94194), human PAK6 (GenBank Accession Numbers NP_{—}064553 and AAF82800), human PAK7 (GenBank Accession Number Q9P286), C. elegans PAK (GenBank Accession Number BAA11844), D. melanogaster PAK (GenBank Accession Number AAC47094), and rat PAK1 (GenBank Accession Number AAB95646). Representative examples of PAK genes encoding PAK proteins include, but are not limited to, human PAK1 (GenBank Accession Number U24152), human PAK2 (GenBank Accession Number U24153), human PAK3 (GenBank Accession Number AF068864), human PAK-4 (GenBank Accession Number AJ011855), human PAK5 (GenBank Accession Number AB040812), and human PAK6 (GenBank Accession Number AF276893). In some embodiments, a PAK polypeptide comprises an amino acid sequence that is at least 70% to 100% identical, e.g., at least 75%, 80%, 85%, 86%, 87%, 88%, 90%, 91%, 92%, 94%, 95%, 96%, 97%, 98%, or any other percent from about 70% to about 100% identical to sequences of GenBank Accession Numbers AAA65441, AAA65442, AAC36097, NP_{—}005875, CAA09820, CAC18720, BAA94194, NP_{—}064553, AAF82800, Q9P286, BAA11844, AAC47094, and/or AAB95646.

In some embodiments, a PAK gene comprises a nucleotide sequence that is at least 70% to 100% identical, e.g., at least 75%, 80%, 85%, 86%, 87%, 88%, 90%, 91%, 92%, 94%, 95%, 96%, 97%, 98%, or any other percent from about 70% to about 100% identical to sequences of GenBank Accession Numbers U24152, U24153, AF068864, AJ011855, AB040812, and/or AF276893.

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent homology between the two sequences is a function of the number of identical positions shared by the sequences (i.e., % identity=# of identical positions/total # of positions (e.g., overlapping positions)×100). In one embodiment the two sequences are the same length.

To determine percent homology between two sequences, the algorithm of Karlin and Altschul (1990) Proc. Natl. Acad. Sci. USA 87:2264-2268, modified as in Karlin and Altschul (1993) Proc. Natl. Acad. Sci. USA 90:5873-5877 is used. Such an algorithm is incorporated into the NBLAST and XBLAST programs of Altschul, et al. (1990) J. Mol. Biol. 215:403-410. BLAST nucleotide searches are performed with the NBLAST program, score=100, wordlength=12 to obtain nucleotide sequences homologous to a nucleic acid molecules described or disclose herein. BLAST protein searches are performed with the XBLAST program, score=50, wordlength=3. To obtain gapped alignments for comparison purposes, Gapped BLAST is utilized as described in Altschul et al. (1997) Nucleic Acids Res. 25:3389-3402. When utilizing BLAST and Gapped BLAST programs, the default parameters of the respective programs (e.g., XBLAST and NBLAST) are used. See the website of the National Center for Biotechnology Information for further details (on the world wide web at ncbi.nlm.nih.gov). Proteins suitable for use in the methods described herein also includes proteins having between 1 to 15 amino acid changes, e.g., 1, 2, 3, 4, 5, 6, 7, 8, 9, 10, 11, 12, 13, 14, or 15 amino acid substitutions, deletions, or additions, compared to the amino acid sequence of any protein PAK activator described herein. In other embodiments, the altered amino acid sequence is at least 75% identical, e.g., 77%, 80%, 82%, 85%, 88%, 90%, 92%, 95%, 97%, 98%, 99%, or 100% identical to the amino acid sequence of any protein PAK activator described herein. Such sequence-variant proteins are suitable for the methods described herein as long as the altered amino acid sequence retains sufficient biological activity to be functional in the compositions and methods described herein. Where amino acid substitutions are made, the substitutions should be conservative amino acid substitutions. Among the common amino acids, for example, a “conservative amino acid substitution” is illustrated by a substitution among amino acids within each of the following groups: (1) glycine, alanine, valine, leucine, and isoleucine, (2) phenylalanine, tyrosine, and tryptophan, (3) serine and threonine, (4) aspartate and glutamate, (5) glutamine and asparagine, and (6) lysine, arginine and histidine. The BLOSUM62 table is an amino acid substitution matrix derived from about 2,000 local multiple alignments of protein sequence segments, representing highly conserved regions of more than 500 groups of related proteins (Henikoff et al (1992), Proc. Natl. Acad. Sci. USA, 89:10915-10919). Accordingly, the BLOSUM62 substitution frequencies are used to define conservative amino acid substitutions that may be introduced into the amino acid sequences described or disclosed herein. Although it is possible to design amino acid substitutions based solely upon chemical properties (as discussed above), the language “conservative amino acid substitution” preferably refers to a substitution represented by a BLOSUM62 value of greater than −1. For example, an amino acid substitution is conservative if the substitution is characterized by a BLOSUM62 value of 0, 1, 2, or 3. According to this system, preferred conservative amino acid substitutions are characterized by a BLOSUM62 value of at least 1 (e.g., 1, 2 or 3), while more preferred conservative amino acid substitutions are characterized by a BLOSUM62 value of at least 2 (e.g., 2 or 3).

As used herein, the term “PAK activity,” unless otherwise specified, includes, but is not limited to, at least one of PAK protein-protein interactions, PAK phosphotransferase activity (intermolecular or intermolecular), translocation, etc. of one or more PAK isoforms.

As used herein, a “PAK activator,” refers to any molecule, compound, or composition that increases PAK activity directly or indirectly.

As used herein, a “direct PAK activator,” refers to: a compound or composition capable of binding to or chemically modifying a PAK so as to increase the PAK's activity level (e.g., a small molecule compound, including, e.g., GTPase, a lipid, a fatty acid, lysophosphatidic acid or a lysophosphatidic acid derivative, sphingosine (2-amino-4-octadecene-1,3-diol) or a sphingosine derivative); a composition that possesses intrinsic PAK activity, e.g., a recombinant PAK, a catalytically active PAK fragment, or a constitutively active PAK mutant isoform such as a PAK3 comprising a (T421E) substitution (see, e.g., Zhang et al. (2005), J Neurosci, 25(13):3379-3388); or a composition comprising a nucleic acid that encodes a polypeptide having PAK activity (e.g., an AAV vector encoding PAK1), or induces the expression of a polypeptide having PAK activity e.g., a zinc finger protein activator of PAK expression.

As used herein, an “indirect PAK activator,” refers to any compound or composition that acts through a signaling pathway that results in a net increase in the activity of one or more PAK isoforms, or alternatively, acts on a downstream effector of PAK, e.g., LIM kinase or myosin light chain kinase.

A “subject” or an “individual,” as used herein, is an animal, for example, a human patient. In some embodiments a “subject” or an “individual” is a human. In some embodiments, the subject suffers from schizophrenia, clinical depression, epilepsy, or age-related cognitive decline.

In some embodiments, a pharmacological composition comprising a PAK activator is “administered peripherally” or “peripherally administered.” As used herein, these terms refer to any form of administration of an agent, e.g., a therapeutic agent, to an individual that is not direct administration to the CNS, i.e., that brings the agent in contact with the non-brain side of the blood-brain barrier. “Peripheral administration,” as used herein, includes intravenous, intra-arterial, subcutaneous, intramuscular, intraperitoneal, transdermal, by inhalation, transbuccal, intranasal, rectal, oral, parenteral, sublingual, or trans-nasal. In some embodiments, a PAK activator is administered by an intracerebral route.

The terms “polypeptide,” and “protein” are used interchangeably herein to refer to a polymer of amino acid residues. That is, a description directed to a polypeptide applies equally to a description of a protein, and vice versa. The terms apply to naturally occurring amino acid polymers as well as amino acid polymers in which one or more amino acid residues is a non-naturally occurring amino acid, e.g., an amino acid analog. As used herein, the terms encompass amino acid chains of any length, including full length proteins (i.e., antigens), wherein the amino acid residues are linked by covalent peptide bonds.

The term “amino acid” refers to naturally occurring and non-naturally occurring amino acids, as well as amino acid analogs and amino acid mimetics that function in a manner similar to the naturally occurring amino acids. Naturally encoded amino acids are the 20 common amino acids (alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine) and pyrolysine and selenocysteine. Amino acid analogs refers to compounds that have the same basic chemical structure as a naturally occurring amino acid, i.e., an a carbon that is bound to a hydrogen, a carboxyl group, an amino group, and an R group, such as, homoserine, norleucine, methionine sulfoxide, methionine methyl sulfonium. Such analogs have modified R groups (such as, norleucine) or modified peptide backbones, but retain the same basic chemical structure as a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The term “nucleic acid” refers to deoxyribonucleotides, deoxyribonucleosides, ribonucleosides, or ribonucleotides and polymers thereof in either single- or double-stranded form. Unless specifically limited, the term encompasses nucleic acids containing known analogues of natural nucleotides which have similar binding properties as the reference nucleic acid and are metabolized in a manner similar to naturally occurring nucleotides. Unless specifically limited otherwise, the term also refers to oligonucleotide analogs including PNA (peptidonucleic acid), analogs of DNA used in antisense technology (phosphorothioates, phosphoroamidates, and the like). Unless otherwise indicated, a particular nucleic acid sequence also implicitly encompasses conservatively modified variants thereof (including but not limited to, degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Cassol et al. (1992); Rossolini et al., Mol. Cell. Probes 8:91-98 (1994)).

The terms “isolated” and “purified” refer to a material that is substantially or essentially removed from or concentrated in its natural environment. For example, an isolated nucleic acid is one that is separated from the nucleic acids that normally flank it or other nucleic acids or components (proteins, lipids, etc. . . . ) in a sample. In another example, a polypeptide is purified if it is substantially removed from or concentrated in its natural environment. Methods for purification and isolation of nucleic acids and proteins are documented methodologies.

The term “antibody” describes an immunoglobulin whether natural or partly or wholly synthetically produced. The term also covers any polypeptide or protein having a binding domain which is, or is homologous to, an antigen-binding domain. CDR grafted antibodies are also contemplated by this term.

The term antibody as used herein will also be understood to mean one or more fragments of an antibody that retain the ability to specifically bind to an antigen, (see generally, Holliger et al., Nature Biotech. 23 (9) 1126-1129 (2005)). Non-limiting examples of such antibodies include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544 546), which consists of a VH domain; and (vi) an isolated complementarity determining region (CDR). Furthermore, although the two domains of the Fv fragment, VL and VH, are coded for by separate genes, they are optionally joined, using recombinant methods, by a synthetic linker that enables them to be made as a single protein chain in which the VL and VH regions pair to form monovalent molecules (known as single chain Fv (scFv); see e.g., Bird et al. (1988) Science 242:423 426; and Huston et al. (1988) Proc. Natl. Acad. Sci. USA 85:5879 5883; and Osbourn et al. (1998) Nat. Biotechnol. 16:778). Such single chain antibodies are also intended to be encompassed within the term antibody. Any VH and VL sequences of specific scFv is optionally linked to human immunoglobulin constant region cDNA or genomic sequences, in order to generate expression vectors encoding complete IgG molecules or other isotypes. VH and VL are also optionally used in the generation of Fab, Fv or other fragments of immunoglobulins using either protein chemistry or recombinant DNA technology. Other forms of single chain antibodies, such as diabodies are also encompassed.

“F(ab′)2” and “Fab” moieties are optionally produced by treating immunoglobulin (monoclonal antibody) with a protease such as pepsin and papain, and includes an antibody fragment generated by digesting immunoglobulin near the disulfide bonds existing between the hinge regions in each of the two H chains. For example, papain cleaves IgG upstream of the disulfide bonds existing between the hinge regions in each of the two H chains to generate two homologous antibody fragments in which an L chain composed of VL (L chain variable region) and CL (L chain constant region), and an H chain fragment composed of VH (H chain variable region) and CHγ1 (γ1 region in the constant region of H chain) are connected at their C terminal regions through a disulfide bond. Each of these two homologous antibody fragments is called Fab′. Pepsin also cleaves IgG downstream of the disulfide bonds existing between the hinge regions in each of the two H chains to generate an antibody fragment slightly larger than the fragment in which the two above-mentioned Fab′ are connected at the hinge region. This antibody fragment is called F(ab′)2.

The Fab fragment also contains the constant domain of the light chain and the first constant domain (CH1) of the heavy chain. Fab′ fragments differ from Fab fragments by the addition of a few residues at the carboxyl terminus of the heavy chain CH1 domain including one or more cysteine(s) from the antibody hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteines between them. Other chemical couplings of antibody fragments are documented.

“Fv” is the minimum antibody fragment which contains a complete antigen-recognition and antigen-binding site. This region consists of a dimer of one heavy chain and one light chain variable domain in tight, non-covalent association. It is in this configuration that the three hypervariable regions of each variable domain interact to define an antigen-binding site on the surface of the VH-VL dimer. Collectively, the six hypervariable regions confer antigen-binding specificity to the antibody. However, even a single variable domain (or half of an Fv comprising only three hypervariable regions specific for an antigen) has the ability to recognize and bind antigen, although at a lower affinity than the entire binding site.

“Single-chain Fv” or “sFv” antibody fragments comprise a VH, a VL, or both a VH and VL domain of an antibody, wherein both domains are present in a single polypeptide chain. In some embodiments, the Fv polypeptide further comprises a polypeptide linker between the VH and VL domains which enables the sFv to form the desired structure for antigen binding. For a review of sFv see, e.g., Pluckthun in The Pharmacology of Monoclonal Antibodies, Vol. 113, Rosenburg and Moore eds. Springer-Verlag, New York, pp. 269 315 (1994).

A “chimeric” antibody includes an antibody derived from a combination of different mammals. The mammal is, for example, a rabbit, a mouse, a rat, a goat, or a human. The combination of different mammals includes combinations of fragments from human and mouse sources.

In some embodiments, an antibody described or disclosed herein is a monoclonal antibody (MAb), typically a chimeric human-mouse antibody derived by humanization of a mouse monoclonal antibody. Such antibodies are obtained from, e.g., transgenic mice that have been “engineered” to produce specific human antibodies in response to antigenic challenge. In this technique, elements of the human heavy and light chain locus are introduced into strains of mice derived from embryonic stem cell lines that contain targeted disruptions of the endogenous heavy chain and light chain loci. In some embodiments, the transgenic mice synthesize human antibodies specific for human antigens, and the mice are used to produce human antibody-secreting hybridomas.

### BRIEF DESCRIPTION OF FIGURE

FIG. 1. Dendritic spine shapes

### DETAILED DESCRIPTION OF THE INVENTION

A number of NCs are characterized by abnormal dendritic spine morphology, spine density, spine size, spine plasticity, spine motility, and/or low spine density as described in a number of studies referred to herein. On the other hand PAK kinase activity has been implicated in spine morphogenesis, maturation, and maintenance. See, e.g., Kreis et al (2007), J Biol Chem, 282(29):21497-21506; Zhao et al (2006), Nat Neurosci, 9(2):234-242; Zhang et al (2005), J Neurosci, 25(31):3379-3388 Ethell et al (2005), Prog in Neurobiol, 75:161-205; Hayashi et al (2004), Neuron, 42(5):773-787; Penzes et al (2003), Neuron, 37:263-274. Thus, in the methods for treating NCs described herein PAK activity is stimulated by administering a PAK activator to rescue defects in spine morphology, size, plasticity, and/or density associated with NCs as described herein. NCs that are treated by the methods described herein include, but are not limited to, psychotic disorders, mood disorders, age-related cognitive decline, epilepsy, Huntington's disease, Down's syndrome, Niemann-Pick disease, spongiform encephalitis, Lafora disease, Maple syrup urine disease, maternal phenylketonuria, atypical phenylketonuria, and tuberous sclerosis. Symptoms and diagnostic criteria for NCs are described in detail in the Diagnostic and Statistical Manual of Mental Disorders, fourth edition, American Psychiatric Association (2005) (DSM-IV).

Abnormal dendritic spine morphology, size, plasticity, and/or density have been found in a number of NCs as described below. Accordingly, in some embodiments, the methods described herein are used to treat a subject suffering from a neuropsychiatric condition, other than Alzheimer's disease or Fragile-X Mental Retardation, that is associated with an abnormal (e.g., lower than normal) dendritic spine density, a reduction in spine size, a reduction in spine plasticity, defective spine morphology, a reduction in spine plasticity, or a reduction in spine motility. In some embodiments, the methods described herein are used to treat a subject suffering from a psychotic disorder. Examples of psychotic disorders include, but are not limited to, schizophrenia, schizoaffective disorder, schizophreniform disorder, brief psychotic disorder, delusional disorder, shared psychotic disorder (Folie a Deux), substance induced psychosis, and psychosis due to a general medical condition. See, e.g., Black et al. (2004), Am J Psychiatry, 161:742-744; Broadbelt et al. (2002), Schizophr Res, 58:75-81; Glantz et al. (2000), Arch Gen Psychiatry 57:65-73; and Kalus et al. (2000), Neuroreport, 11:3621-3625.

In some embodiments, the methods described herein are used to treat a subject suffering from a mood disorder. Examples of mood disorders include, but are not limited to, clinical depression, bipolar disorder, cyclothymia, and dysthymia. See, e.g., Hajszan et al (2005), Eur J Neurosci, 21:1299-1303; Law et al (2004) Am J Psychiatry, 161(10):1848-1855; Norrholm et al. (2001), Synapse, 42:151-163; and Rosoklija et al., (2000), Arch Gen Psychiatry, 57:349-356.

In some embodiments, the methods described herein are used to treat a subject suffering from age-related cognitive decline. See, e.g., Dickstein et al (2007), Aging Cell, 6:275-284; and Page et al. (2002), Neuroscience Letters, 317:37-41.

In some embodiments, the methods described herein are used to treat a subject suffering from epilepsy. See, e.g., Wong (2005), Epilepsy and Behavior, 7:569-577; Swann et al (2000), Hippocampus, 10:617-625; and Jiang et al (1998), J Neurosci, 18(20):8356-8368.

In some embodiments, the methods described herein are used to treat a subject suffering from Parkinson's Disease or Huntington's Disease. See, e.g., Neely et al (2007), Neuroscience, 149(2):457-464; Spires et al (2004), Eur J Neurosci, 19:2799-2807; Klapstein et al (2001), J Neurophysiol, 86:2667-2677; Ferrante et al (1991), J Neurosci, 11:3877-3887; and Graveland et al (1985), Science, 227:770-773.

In some embodiments, the methods described herein are used to treat a subject suffering from Down's syndrome, Niemann-Pick disease, spongiform encephalitis, Lafora disease, Maple syrup urine disease, maternal phenylketonuria, atypical phenylketonuria, and tuberous sclerosis. In some embodiments, a composition containing a therapeutically effective amount of a PAK activator is administered prophylactically to a subject that while not overtly manifesting symptoms of a NC has been identified as having a high risk of developing a NC, e.g., the subject is identified as being a carrier of a polymorphism associated with clinical depression (see, e.g., Hashimoto et al (2006), Hum Mol Genet, 15(20):3024-3033 or schizophrenia (see, e.g., Hall et al (2006), Nat Neurosci., 9(12):1477-8, or the subject is from a family that has a high incidence of a particular NC. In some embodiments, MRI is used to detect brain morphological changes in children prior to the onset of schizophrenia (see, e.g., Toga et al (2006), TINS, 29(3):148-159). For some NCs, risk is age-dependent. For example, the typical age of onset for schizophrenia is between 20-28 for males and 26-32 for females. Accordingly, in some embodiments, a PAK activator is administered to a subject at risk between about 1 to about 10 years, e.g., 1, 2, 3, 4, 5, 6, 7, 8, 9, or 10 years prior to an established age range of onset for a particular NC.

p21-Activated Kinases (PAKs)

The PAKs constitute a family of serine-threonine kinases that can be separated into two groups: Group I PAKs, PAK1-PAK3 and Group II PAKs, PAK4-PAK6. See, e.g., Zhao et al. (2005), Biochem J, 386:201-214. These kinases function downstream of the small GTPases Rac and/or Cdc42 to regulate multiple cellular functions, including dendritic morphogenesis and maintenance (see, e.g., Ethel et al (2005), Prog in Neurobiol, 75:161-205; Penzes et al (2003), Neuron, 37:263-274), motility, morphogenesis, angiogenesis, and apoptosis, (see, e.g., Bokoch et al., 2003, Annu. Rev. Biochem., 72:743; and Hofmann et al., 2004, J. Cell Sci., 117:4343;). GTP-bound Rac and/or Cdc42 bind to inactive PAK, releasing steric constraints imposed by a PAK autoinhibitory domain and/or permitting PAK auto-phosphorylation and/or activation. Numerous autophosphorylation sites have been identified that serve as markers for activated PAK.

Prominent upstream effectors of PAK include, but are not limited to small GTPases including Cdc42, Rac, TC10, CHP and Wrch-1, TrkB receptors, NMDA receptors, EphB receptors, FMRP, p-21-activated kinase interacting exchange factor (PIX), G-protein-coupled receptor kinase-interacting protein 1 (GIT1), Kalirin-7, Tiam1, caspase 3, sphinogosine and 3-phosphoinositide-dependent-kinase-1 (PDK1). See, e.g., Zhao et al. (2005), Biochem J, 386:201-214.

Prominent downstream targets of mammalian PAK include, but are not limited to, substrates of PAK kinase, such as Myosin light chain kinase (MLCK), regulatory Myosin light chain (R-MLC), Myosins I heavy chain, myosin II heavy chain, Myosin VI, Caldesmon, Desmin, Op18/stathmin, Merlin, Filamin A, LIM kinase (LIMK), Ras, Raf, Mek, p47^{phox}, BAD, caspase 3, estrogen and/or progesterone receptors, RhoGEF, GEF-H1, NET1, Gaz, phosphoglycerate mutase-B, RhoGDI prolactin, p41^{Arc}, and/or Aurora-A (See, e.g., Bokoch et al., 2003, Annu. Rev. Biochem., 72:743; and Hofmann et al., 2004, J. Cell Sci., 117:4343). Other substances that bind to PAK in cells include CIB; sphingolipids; lysophosphatidic acid, G-protein β and/or γ subunits; PIX/COOL; GIT/PKL; Nef; Nef; Paxillin; NESH; SH3-containing proteins (e.g. Nck and/or Grb2); kinases (e.g. Akt, PDK1, PI 3-kinase/p85, Cdk5, Cdc2, Src kinases, Abl, and/or protein kinase A (PKA)); and/or phosphatases (e.g. phosphatase PP2A, POPX1, and/or POPX2).

### PAK Activators

As described herein, a subject suffering from a NC is treated by administration of a pharmaceutical composition containing a PAK activator. In some embodiments, a PAK activator is a PAK1 activator. In some embodiments, a PAK activator is a PAK2 activator. In some embodiments, a PAK activator is a PAK3 activator. In some embodiments, a PAK activator is a PAK4 activator. In some embodiments, a PAK activator is a PAK5 activator. In some embodiments, a PAK activator is a PAK6 activator.

In some embodiments, a PAK activator is a direct PAK activator.

In some embodiments, a direct PAK activator is a constitutively active form of a PAK (CA-PAK), e.g., a CA-PAK1, CA-PAK2, CA-PAK3, CA-PAK4, CA-PAK5, or a CA-PAK6. In some embodiments, a CA-PAK is a CA-PAK1 (T421E) or a CA-PAK3 (T421E) (Zhang et al (2005), J Neurosci, 25(13):3379-3388. In some embodiments, the CA-PAK is PAK 3b, a constitutively active alternative splice form of PAK3 (Rousseau et al (2003), J Biol Chem, 278(6):3912-3920). In alternative embodiments, the ratio of endogenous expression of the constitutively active PAK3b isoform to the regulated PAK3a isoform is increased by mRNA splicing redirection, e.g., administering peptide nucleic acids (PNA) or phosphorodiamidate morpholino oligomers (PMO) as described in, e.g., Wheeler et al (2007), J Clin Invest, 117(12):3952-3957; Wilton et al (2005), Curr Gene Ther, 5(5):467-483. In other embodiments, the direct PAK activator is TAT-modified peptide comprising the sequence encoded by the PAK3 “b” exon: RKKRRQRRR-G-PDLYGSQMCPGKLPE.

In some embodiments, a CA-PAK is a catalytically active PAK fragment that comprises amino acids 201 to 491, but excludes the regulatory domain (Buchwald et al (2001), Mol Cell Biol, 15:5179-5189. In some embodiments, a CA-PAK is delivered to one or more brain regions of a subject by administration of a CA-PAK viral expression vector, e.g., an AAV vector, a lentiviral vector, an adenoviral vector, or a HSV vector. A number of viral vectors for delivery of therapeutic proteins are described in, e.g., U.S. Pat. Nos. 7,244,423, 6,780,409, 5,661,033. In some embodiments, indirect activators of PAK act by increasing transcription or translation, or by reducing mRNA or protein turnover of one or more PAK isoforms. In some embodiments, a CA-PAK to be expressed is under the control of an inducible promoter (e.g., a promoter containing a tet-operator). Inducible viral expression vectors are known. See, e.g., U.S. Pat. No. 6,953,575. Inducible expression of a CA-PAK allows for tightly controlled and reversible increases of CA-PAK expression by varying the dose of an inducing agent (e.g., tetracycline) administered to the subject.

In other embodiments, a direct PAK activator is a small molecule. As referred to herein, a “small molecule” is an organic molecule that is less than about 5 kilodaltons (kDa) in size. In some embodiments, the small molecule is less than about 4 kDa, 3 kDa, about 2 kDa, or about 1 kDa. In some embodiments, the small molecule is less than about 800 daltons (Da), about 600 Da, about 500 Da, about 400 Da, about 300 Da, about 200 Da, or about 100 Da. In some embodiments, a small molecule is less than about 4000 g/mol, less than about 3000 g/mol, 2000 g/mol, less than about 1500 g/mol, less than about 1000 g/mol, less than about 800 g/mol, or less than about 500 g/mol. In some embodiments, small molecules are non-polymeric. Typically, small molecules are not proteins, polypeptides, polynucleotides, oligonucleotides, polysaccharides, glycoproteins, or proteoglycans. A derivative of a small molecule refers to a molecule that shares the same structural core as the original small molecule, but which is prepared by a series of chemical reactions from the original small molecule. As one example, a pro-drug of a small molecule is a derivative of that small molecule. An analog of a small molecule refers to a molecule that shares the same or similar structural core as the original small molecule, and which is synthesized by a similar or related route, or art-recognized variation, as the original small molecule.

In some embodiments, a small molecule direct PAK activator is a lipid, lipid metabolite, or fatty acid. In some embodiments, a PAK activator is sphingosine (2-amino-4-octadecene-1,3-diol) or a sphingosine derivative. Sphingosine has been to shown to activate PAK1 independently of GTPase activators such as Cdc42 or Rac (Bokoch et al (1998), J Biol Chem, 273(14):8137-8144). In some embodiments, a small molecule direct PAK activator is lysophosphatidic acid.

In some embodiments, a direct PAK activator is a reversible PAK activator. In other embodiments, a direct PAK activator is an irreversible PAK activator. Direct PAK activators are optionally used for the manufacture of a medicament for treating any of the NCs described herein (e.g., psychotic disorders, mood disorders, age-related cognitive decline, epilepsy, Huntington's Disease, or Parkinson's Disease).

### Identification and Characterization of Direct PAK Activators

Small molecule direct PAK activators are optionally identified in high-throughput in vitro or cellular assays as described in, e.g., Yu et al (2001), J Biochem (Tokyo); 129(2):243-251; Rininsland et al (2005), BMC Biotechnol, 5:16; and Allen et al (2006), ACS Chem Biol; 1(6):371-376. PAK activators suitable for the methods described herein are available from a variety of sources including both natural (e.g., plant extracts) and synthetic. For example, candidate PAK activators are isolated from a combinatorial library, i.e., a collection of diverse chemical compounds generated by either chemical synthesis or biological synthesis by combining a number of chemical “building blocks.” For example, a linear combinatorial chemical library such as a polypeptide library is formed by combining a set of chemical building blocks called amino acids in every possible way for a given compound length (i.e., the number of amino acids in a polypeptide compound). Millions of chemical compounds can be synthesized through such combinatorial mixing of chemical building blocks, as desired. Theoretically, the systematic, combinatorial mixing of 100 interchangeable chemical building blocks results in the synthesis of 100 million tetrameric compounds or 10 billion pentameric compounds. See Gallop et al. (1994), J. Med. Chem. 37(9), 1233. Each member of a library may be singular and/or may be part of a mixture (e.g. a “compressed library”). The library may comprise purified compounds and/or may be “dirty” (i.e., containing a quantity of impurities). Preparation and screening of combinatorial chemical libraries are documented methodologies. See Cabilly, ed., Methods in Molecular Biology, Humana Press, Totowa, N.J., (1998). Combinatorial chemical libraries include, but are not limited to: diversomers such as hydantoins, benzodiazepines, and dipeptides, as described in, e.g., Hobbs et al. (1993), Proc. Natl. Acad. Sci. U.S.A. 90, 6909; analogous organic syntheses of small compound libraries, as described in Chen et al. (1994), J. Amer. Chem. Soc., 116: 2661; Oligocarbamates, as described in Cho, et al. (1993), Science 261, 1303; peptidyl phosphonates, as described in Campbell et al. (1994), J. Org. Chem., 59: 658; and small organic molecule libraries containing, e.g., thiazolidinones and metathiazanones (U.S. Pat. No. 5,549,974), pyrrolidines (U.S. Pat. Nos. 5,525,735 and 5,519,134), benzodiazepines (U.S. Pat. No. 5,288,514). In addition, numerous combinatorial libraries are commercially available from, e.g., ComGenex (Princeton, N.J.); Asinex (Moscow, Russia); Tripos, Inc. (St. Louis, Mo.); ChemStar, Ltd. (Moscow, Russia); 3D Pharmaceuticals (Exton, Pa.); and Martek Biosciences (Columbia, Md.).

Devices for the preparation of combinatorial libraries are commercially available (see, e.g., 357 MPS, 390 MPS from Advanced Chem Tech, Louisville, Ky.; Symphony from Rainin, Woburn, Mass.; 433A from Applied Biosystems, Foster City, Calif.; and 9050 Plus from Millipore, Bedford, Mass.). A number of robotic systems have also been developed for solution phase chemistries. These systems include automated workstations like the automated synthesis apparatus developed by Takeda Chemical Industries, LTD (Osaka, Japan), and many robotic systems utilizing robotic arms (Zymate II, Zymark by a chemist. Any of the above devices are optionally used to generate combinatorial libraries for identification and characterization Corporation, Hopkinton, Mass.; Orca, Hewlett Packard, Palo Alto, Calif.) which mimic the manual synthetic operations performed of small molecule PAK activators suitable for the methods disclosed herein.

The identification of potential direct PAK activators may be determined by, for example, assaying the in vitro kinase activity of PAK in the presence of candidate activators. In such assays, PAK and/or a characteristic PAK fragment produced by recombinant methods is contacted with a substrate in the presence of a phosphate donor (e.g., ATP) containing radiolabeled phosphate, and PAK-dependent incorporation is measured. “Substrate” includes any substance containing a suitable hydroxyl moiety that is capable of accepting the γ-phosphate group from a donor molecule such as ATP in a reaction catalyzed by PAK. In some instances, the substrate is an endogenous substrate of PAK, i.e. a naturally occurring substance that is phosphorylated in unmodified cells by naturally-occurring PAK or any other substance that is not normally phosphorylated by PAK in physiological conditions, but is optionally phosphorylated in the employed conditions. In some instances, the substrate is a protein or a peptide, and the phosphorylation reaction may occur on a serine and/or threonine residue of the substrate. For example, specific substrates, which are commonly employed in such assays include, but are not limited to, histone proteins and myelin basic protein.

In some embodiments, the detection of PAK dependent phosphorylation of a substrate is quantified in any suitable manner including methods other than measurement of radiolabeled phosphate incorporation. For example, quantitation methods include measurement or detection of physiochemical properties of the substrate, such as electrophoretic mobility, chromatographic properties, light absorbance, fluorescence, phosphorescence and the like. In certain embodiments, by way of non-limiting examples, monoclonal or polyclonal antibodies are generated which selectively recognize phosphorylated forms of the substrate from non-phosphorylated forms thereby allowing antibodies to function as indicators of PAK kinase activity.

In some embodiments, high-throughput PAK kinase assays are performed in, for example, microtiter plates with each well containing PAK kinase or an active fragment thereof, substrate covalently linked to each well, P³² radiolabeled ATP and a potential PAK activator candidate. Microtiter plates can contain any number of wells, e.g., 96 wells or 1536 wells, for large scale screening of combinatorial library compounds. After the phosphorylation reaction has completed, the plates are washed leaving the bound substrate. In some instances, the plates are then read on a detector (e.g., an absorbance detector) for phosphate group incorporation via autoradiography or antibody detection. In certain embodiments, candidate PAK activators are identified by their ability to increase the amount of PAK phosphotransferase ability upon a substrate in comparison with PAK phosphotransferase ability alone.

In some embodiments, a direct PAK activator suitable for the methods described herein increases PAK activity relative to a basal level of PAK activity by about 1.1 fold to about 100 fold, e.g., to about 1.2 fold, 1.5 fold, 1.6 fold, 1.7 fold, 2.0 fold, 3.0 fold, 5.0 fold, 6.0 fold, 7.0 fold, 8.5 fold, 9.7 fold, 10 fold, 12 fold, 14 fold, 15 fold, 20 fold, 30 fold, 40 fold, 50 fold, 60 fold, 70 fold, 90 fold, 95 fold, or by any other amount from about 1.1 fold to about 100 fold relative to basal PAK activity.

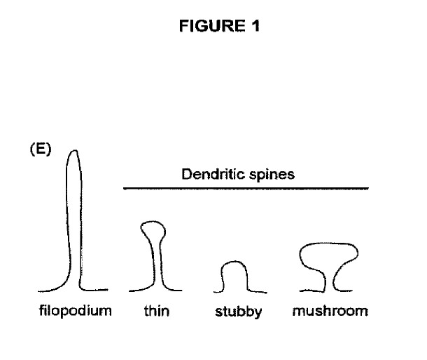

In some embodiments, a PAK activator suitable for the methods described herein modulates a spine:head ratio, e.g., ratio of the volume of the spine to the volume of the head, ratio of the length of a spine to the length of a head of the spine, ratio of the surface area of a spine to the surface area of the head of a spine, or the like, compared to a spine:head ratio in the absence of a PAK activator. In certain embodiments, a PAK activator suitable for the methods described herein modulates the volume of the spine head, the width of the spine head, the surface area of the spine head, the length of the spine shaft, the diameter of the spine shaft, or a combination thereof. In some embodiments, provided herein is a method of modulating the volume of a spine head, the width of a spine head, the surface area of a spine head, the length of a spine shaft, the diameter of a spine shaft, or a combination thereof, by contacting a neuron comprising the dendritic spine with an effective amount of a PAK activator described herein. In specific embodiments, the neuron is contacted with the PAK activator in vivo. FIG. 1 illustrates various morphologies of the dendritic spines.

Changes in spine morphology are detected using any suitable method, e.g., by use of 3D and/or 4D real time interactive imaging and visualization. In some instances, the Imaris suite of products (available from Bitplane Scientific Solutions) provides functionality for visualization, segmentation and interpretation of 3D and 4D microscopy datasets obtained from confocal and wide field microscopy data.

In some embodiments, a direct PAK activator used for the methods described herein has in vitro ED₅₀ for PAK activation of Less than 100 μM (e.g., less than 10 μM, less than 5 μM, less than 4 μM, less than 3 μM, less than 1 μM, less than 0.8 μM, less than 0.6 μM, less than 0.5 μM, less than 0.4 μM, less than 0.3 μM, less than less than 0.2 μM, less than 0.1 μM, less than 0.08 μM, less than 0.06 μM, less than 0.05 μM, less than 0.04 μM, less than 0.03 μM, less than less than 0.02 μM, less than 0.01 μM, less than 0.0099 μM, less than 0.0098 μM, less than 0.0097 μM, less than 0.0096 μM, less than 0.0095 μM, less than 0.0094 μM, less than 0.0093 μM, less than 0.00092, or less than 0.0090 μM).

### Indirect PAK Activators

In some embodiments, a NC is treated by administering a pharmacological composition containing a therapeutically effective amount of an agent that activates a signaling pathway that increases PAK activity, or, alternatively, to activate activity of a downstream effector of PAK, e.g., LIM kinase.

In some embodiments, an indirect PAK activator is an agonist of the TrkB receptor, which induces activation of PAK3, a brain-specific isoform of PAK (see, e.g., Rex et al (2007), J Neurosci, 27(10:3017-3029). In some embodiments, the TrkB receptor agonist is Brain-Derived Neurotrophic Factor (BDNF), i.e., the primary naturally occurring ligand of the TrkB receptor. Methods for production of purified recombinant BDNF are described in, e.g., U.S. Pat. No. 5,438,121. In some embodiments, the TrkB agonist is a bifunctional fusion protein comprising BDNF fused to an antibody against a transporter protein expressed on the blood brain barrier (BBB), e.g., an insulin receptor. The BBB has specific receptors, including insulin receptors, that allow the transport from the blood to the brain of several macromolecules. In particular, insulin receptors are suitable as transporters for the BDNF-insulin receptor antibody fusion proteins. Such bifunctional BDNF fusion proteins bind to the extracellular domain (ECD) of the human insulin receptor and are thereby readily transported into the brain from peripheral circulation. Thus, BBB-permeable BDNF fusion proteins are administered peripherally, e.g., by intravenous administration, and yet penetrate into brain tissue to effect activation of TrkB receptors and PAK. Such fusion proteins and methods for their administration are described in detail in U.S. patent application Ser. No. 11/245,546. In some embodiments, a viral vector is administered to increase BDNF levels in one or more brain regions of a subject suffering from an NC. Examples of a viral BDNF expression expression vector are disclosed in, e.g., U.S. Pat. No. 7,244,423. In some embodiments, the Trk B agonist is a TrkB agonist antibody as described in, e.g., U.S. patent application Ser. No. 11/446,875. In some embodiments, the TrkB agonist is a peptide mimetic of BDNF as described in, e.g., O'Leary et al., (2003), J Biol Chem, 278(28):25738-25744.

In some embodiments, an indirect PAK activator is an agonist of Ephrin B (EphB) receptors, which have been shown to induce activation of PAK in hippocampal and cortical neuronal cultures (see, e.g., Penzes et al (2003), Neuron, 37:263-274) and to be critical for spine morphogenesis (Henkemeyer et al (2003), J Cell Biol, 163(6):1313-1326. In some embodiments, the EphB receptor agonists are soluble agonists that comprise the extracellular domain of an Ephrin family ligand or the extracellular domain of an Eph family receptor fused to the Fc domain of human IgG. For example, an EphrinB 1 fusion protein in which the extracellular domain of the membrane protein is fused to the Fc domain of human IgG is used (see, e.g., Wang, et al (1997), Neuron, 18:383-396). See, for examples of methods Stein, et al, Genes and Dev, 12:667-678 (1998), regarding experiments on responses of cells to clustered Ephrin-B1/Fc fusion proteins. Clustering of these hybrid molecules with anti-human Fc antibodies generates soluble agonists: Ephrin-derived “ligand-bodies” for Eph receptors, and conversely, Eph-derived “receptor bodies” for Ephrins. In some embodiments, the agonist of the EphB2 receptor is an ephrin ligand mimetic peptide as described in, e.g., U.S. patent Ser. No. 10/652,407.

In some embodiments, an indirect PAK activator is a constitutively active form of a GTPase. In some embodiments the constitutively active GTPase is Rac, Cdc42, CHP, TC10 or Wrch-1, all of which activate PAK (see, e.g., Zhao et al (2005), Biochem J, 386:201-214). In some embodiments, the constitutively active Rac is Rac V12 (see, e.g., Zhang et al (2003), J Cell Biol, 161(1):131-142). In some embodiments, the constitutively active Cdc42 is Cdc42V12 (see, e.g., Nakamura et al, Genes Cells, 5(7):571-581).

In some embodiments, an indirect activator of PAK is an activator of PDK1. In some instances an indirect activator of PAK of a P13 kinase is an activator of a PI3 kinase. In certain embodiments, an indirect activator of PAK is an activator of Cdc42. In some instances, an indirect activator of PAK is an activator of Rac/Cdc42 interaction. In some instances, an indirect activator of PAK is an activator of GRB2. In certain embodiments, an indirect activator of PAK is an activator of NCK. In certain embodiments, an indirect activator of PAK is an activator of ETK.

In some embodiments, an indirect activator of PAK is an inhibitor of CDK5. In some embodiments, this CDK5 inhibitor is Roscovitine (2-(1-ethyl-2-hydroyethylamino)-6-benzylamino-9-isopropylpurine). In some embodiment, the CDK5 inhibitor is 3-{4-[4-(3-Chloro-phenylamino)-[1,3,5]triazin-2-yl]-pyridin-2-ylamino}-propan-1-ol. In some embodiment, the CDK5 inhibitor is N-(5-isopropyl-thiazol-2-yl)isobutyramide. In some embodiments, the CDK5 inhibitor is SCH-727965 (2-[1-[3-Ethyl-7-(1-oxidopyridin-3-ylmethylamino)pyrazolo [1,5-a]pyrimidin-5-yl]piperidin-2(S)-yl]ethanol).

In some embodiments, indirect activators of PAK act by decreasing transcription and/or translation of PAK binding partners that inhibit its activation (PAK inhibitory binding partners). In some embodiments, indirect PAK activators affect RNA and/or protein half-life of the PAK inhibitory binding partner, for example, by directly affecting mRNA and/or protein stability. In certain embodiments, indirect PAK activators cause the PAK inhibitory binding partner mRNA and/or protein to be more accessible and/or susceptible to nucleases, proteases, and/or the proteasome. In some embodiments, an indirect PAK activator affecst the processing of mRNAs encoding a PAK inhibitory partner thereby stimulating PAK activity. For example, indirect PAK activators function at the level of pre-mRNA splicing, 5′ end formation (e.g. capping), 3′ end processing (e.g. cleavage and/or polyadenylation), nuclear export, and/or association with the translational machinery and/or ribosomes in the cytoplasm. In some embodiments, indirect PAK activators cause a decrease in the level of mRNA and/or protein of an inhibitory PAK binding partner, the half-life of its mRNA and/or protein by at least about 5%, at least about 10%, at least about 20%, at least about 30%, at least about 40%, at least about 50%, at least about 60%, at least about 80%, at least about 90%, at least about 95%, or substantially 100%. In some embodiments, the PAK inhibitory binding partner to be targeted is Fragile X Mental Retardation Protein (FMRP), which has been shown to bind to bind to PAK1 and inhibit its activity (Hayashi et al (2007), Proc Natl Acad Sci USA, 104(27):11489-11494. In some embodiments, an indirect PAK activator comprises one or more RNAi or antisense oligonucleotides directed against FMRP. In some embodiments, an indirect PAK activator comprises one or more ribozymes directed against FMRP. In some embodiments, an indirect PAK activator is a compound that inhibits the binding of FMRP to PAK1. In some embodiments, an indirect PAK activator is a compound that inhibits the binding of FMRP to PAK mRNA.

### Examples of Pharmaceutical Compositions and Methods of Administration

Pharmaceutical compositions are formulated using one or more physiologically acceptable carriers including excipients and auxiliaries which facilitate processing of the active compounds into preparations which are used pharmaceutically. Proper formulation is dependent upon the route of administration chosen. A summary of pharmaceutical compositions is found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins, 1999).

Provided herein are pharmaceutical compositions that include one or more PAK activators and a pharmaceutically acceptable diluent(s), excipient(s), or carrier(s). In addition, a PAK activator is optionally administered as pharmaceutical compositions in which it is mixed with other active ingredients, as in combination therapy. In some embodiments, the pharmaceutical compositions includes other medicinal or pharmaceutical agents, carriers, adjuvants, such as preserving, stabilizing, wetting or emulsifying agents, solution promoters, salts for regulating the osmotic pressure, and/or buffers. In addition, the pharmaceutical compositions also contain other therapeutically valuable substances.

A pharmaceutical composition, as used herein, refers to a mixture of a PAK activator with other chemical components, such as carriers, stabilizers, diluents, dispersing agents, suspending agents, thickening agents, and/or excipients. The pharmaceutical composition facilitates administration of a PAK activator to an organism. In practicing the methods of treatment or use provided herein, therapeutically effective amounts of a PAK activator are administered in a pharmaceutical composition to a mammal having a condition, disease, or disorder to be treated. Preferably, the mammal is a human. A therapeutically effective amount varies depending on the severity and stage of the condition, the age and relative health of the subject, the potency of a PAK activator used and other factors. A PAK activator is optionally used singly or in combination with one or more therapeutic agents as components of mixtures.

The pharmaceutical formulations described herein are optionally administered to a subject by multiple administration routes, including but not limited to, oral, parenteral (e.g., intravenous, subcutaneous, intramuscular), intranasal, buccal, topical, rectal, or transdermal administration routes. The pharmaceutical formulations described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

The pharmaceutical compositions will include at least one PAK activator, as an active ingredient in free-acid or free-base form, or in a pharmaceutically acceptable salt form. In addition, the methods and pharmaceutical compositions described herein include the use of N-oxides, crystalline forms (also known as polymorphs), as well as active metabolites of these PAK activators having the same type of activity. In some situations, PAK activators exist as tautomers. All tautomers are included within the scope of the compounds presented herein. Additionally, a PAK activator exists in unsolvated as well as solvated forms with pharmaceutically acceptable solvents such as water, ethanol, and the like. The solvated forms of a PAK activators presented herein are also considered to be disclosed herein.

“Carrier materials” include any commonly used excipients in pharmaceutics and should be selected on the basis of compatibility with compounds disclosed herein, such as, a PAK activator, and the release profile properties of the desired dosage form. Exemplary carrier materials include, e.g., binders, suspending agents, disintegration agents, filling agents, surfactants, solubilizers, stabilizers, lubricants, wetting agents, diluents, and the like.

Moreover, the pharmaceutical compositions described herein, which include a PAK activator, are formulated into any suitable dosage form, including but not limited to, aqueous oral dispersions, liquids, gels, syrups, elixirs, slurries, suspensions and the like, for oral ingestion by a patient to be treated, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations.

Pharmaceutical preparations for oral use are optionally obtained by mixing one or more solid excipient with a PAK activator, optionally grinding the resulting mixture, and processing the mixture of granules, after adding suitable auxiliaries, if desired, to obtain tablets or dragee cores. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. If desired, disintegrating agents are added, such as the cross linked croscarmellose sodium, polyvinylpyrrolidone, agar, or alginic acid or a salt thereof such as sodium alginate.

Dragee cores are provided with suitable coatings. For this purpose, concentrated sugar solutions are generally used, which optionally contain gum arabic, talc, polyvinylpyrrolidone, carbopol gel, polyethylene glycol, and/or titanium dioxide, lacquer solutions, and suitable organic solvents or solvent mixtures. Dyestuffs or pigments are optionally added to the tablets or dragee coatings for identification or to characterize different combinations of active compound doses.

In some embodiments, the solid dosage forms disclosed herein are in the form of a tablet, (including a suspension tablet, a fast-melt tablet, a bite-disintegration tablet, a rapid-disintegration tablet, an effervescent tablet, or a caplet), a pill, a powder (including a sterile packaged powder, a dispensable powder, or an effervescent powder) a capsule (including both soft or hard capsules, e.g., capsules made from animal-derived gelatin or plant-derived HPMC, or “sprinkle capsules”), solid dispersion, solid solution, bioerodible dosage form, controlled release formulations, pulsatile release dosage forms, multiparticulate dosage forms, pellets, granules, or an aerosol. In other embodiments, the pharmaceutical formulation is in the form of a powder. In still other embodiments, the pharmaceutical formulation is in the form of a tablet, including but not limited to, a fast-melt tablet. Additionally, pharmaceutical formulations of a PAK activator are optionally administered as a single capsule or in multiple capsule dosage form. In some embodiments, the pharmaceutical formulation is administered in two, or three, or four, capsules or tablets.

In another aspect, dosage forms include microencapsulated formulations. In some embodiments, one or more other compatible materials are present in the microencapsulation material. Exemplary materials include, but are not limited to, pH modifiers, erosion facilitators, anti-foaming agents, antioxidants, flavoring agents, and carrier materials such as binders, suspending agents, disintegration agents, filling agents, surfactants, solubilizers, stabilizers, lubricants, wetting agents, and diluents.

Exemplary microencapsulation materials useful for delaying the release of the formulations including a PAK activator, include, but are not limited to, hydroxypropyl cellulose ethers (HPC) such as Klucel® or Nisso HPC, low-substituted hydroxypropyl cellulose ethers (L-HPC), hydroxypropyl methyl cellulose ethers (HPMC) such as Seppifilm-LC, Pharmacoat®, Metolose SR, Methocel®-E, Opadry YS, PrimaFlo, Benecel MP824, and Benecel MP843, methylcellulose polymers such as Methocel®-A, hydroxypropylmethylcellulose acetate stearate Aqoat (HF-LS, HF-LG, HF-MS) and Metolose®, Ethylcelluloses (EC) and mixtures thereof such as E461, Ethocel®, Aqualon®-EC, Surelease®, Polyvinyl alcohol (PVA) such as Opadry AMB, hydroxyethylcelluloses such as Natrosol®, carboxymethylcelluloses and salts of carboxymethylcelluloses (CMC) such as Aqualon®-CMC, polyvinyl alcohol and polyethylene glycol co-polymers such as Kollicoat IR®, monoglycerides (Myverol), triglycerides (KLX), polyethylene glycols, modified food starch, acrylic polymers and mixtures of acrylic polymers with cellulose ethers such as Eudragit® EPO, Eudragit® L30D-55, Eudragit® FS 30D Eudragit® L100-55, Eudragit® L100, Eudragit® 5100, Eudragit® RD100, Eudragit® E100, Eudragit® L12.5, Eudragit® 512.5, Eudragit® NE30D, and Eudragit® NE 40D, cellulose acetate phthalate, sepifilms such as mixtures of HPMC and stearic acid, cyclodextrins, and mixtures of these materials.

The pharmaceutical solid oral dosage forms including formulations described herein, which include a PAK activator, are optionally further formulated to provide a controlled release of a PAK activator. Controlled release refers to the release of a PAK activator from a dosage form in which it is incorporated according to a desired profile over an extended period of time. Controlled release profiles include, for example, sustained release, prolonged release, pulsatile release, and delayed release profiles. In contrast to immediate release compositions, controlled release compositions allow delivery of an agent to a subject over an extended period of time according to a predetermined profile. Such release rates provide therapeutically effective levels of agent for an extended period of time and thereby provide a longer period of pharmacologic response while minimizing side effects as compared to conventional rapid release dosage forms. Such longer periods of response provide for many inherent benefits that are not achieved with the corresponding short acting, immediate release preparations.

In other embodiments, the formulations described herein, which include a PAK activator, are delivered using a pulsatile dosage form. A pulsatile dosage form is capable of providing one or more immediate release pulses at predetermined time points after a controlled lag time or at specific sites. Pulsatile dosage forms including the formulations described herein, which include a PAK activator, are optionally administered using a variety of pulsatile formulations that include, but are not limited to, those described in U.S. Pat. Nos. 5,011,692, 5,017,381, 5,229,135, and 5,840,329. Other pulsatile release dosage forms suitable for use with the present formulations include, but are not limited to, for example, U.S. Pat. Nos. 4,871,549, 5,260,068, 5,260,069, 5,508,040, 5,567,441 and 5,837,284.

Liquid formulation dosage forms for oral administration are optionally aqueous suspensions selected from the group including, but not limited to, pharmaceutically acceptable aqueous oral dispersions, emulsions, solutions, elixirs, gels, and syrups. See, e.g., Singh et al., Encyclopedia of Pharmaceutical Technology, 2nd Ed., pp. 754-757 (2002). In addition to a PAK activator, the liquid dosage forms optionally include additives, such as: (a) disintegrating agents; (b) dispersing agents; (c) wetting agents; (d) at least one preservative, (e) viscosity enhancing agents, (f) at least one sweetening agent, and (g) at least one flavoring agent. In some embodiments, the aqueous dispersions further includes a crystal-forming inhibitor.

In some embodiments, the pharmaceutical formulations described herein are self-emulsifying drug delivery systems (SEDDS). Emulsions are dispersions of one immiscible phase in another, usually in the form of droplets. Generally, emulsions are created by vigorous mechanical dispersion. SEDDS, as opposed to emulsions or microemulsions, spontaneously form emulsions when added to an excess of water without any external mechanical dispersion or agitation. An advantage of SEDDS is that only gentle mixing is required to distribute the droplets throughout the solution. Additionally, water or the aqueous phase is optionally added just prior to administration, which ensures stability of an unstable or hydrophobic active ingredient. Thus, the SEDDS provides an effective delivery system for oral and parenteral delivery of hydrophobic active ingredients. In some embodiments, SEDDS provides improvements in the bioavailability of hydrophobic active ingredients. Methods of producing self-emulsifying dosage forms include, but are not limited to, for example, U.S. Pat. Nos. 5,858,401, 6,667,048, and 6,960,563.

Suitable intranasal formulations include those described in, for example, U.S. Pat. Nos. 4,476,116, 5,116,817 and 6,391,452. Nasal dosage forms generally contain large amounts of water in addition to the active ingredient. Minor amounts of other ingredients such as pH adjusters, emulsifiers or dispersing agents, preservatives, surfactants, gelling agents, or buffering and other stabilizing and solubilizing agents are optionally present.

For administration by inhalation, a PAK activator is optionally in a form as an aerosol, a mist or a powder. Pharmaceutical compositions described herein are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol, the dosage unit is determined by providing a valve to deliver a metered amount. Capsules and cartridges of, such as, by way of example only, gelatin for use in an inhaler or insufflator are formulated containing a powder mix of a PAK activator and a suitable powder base such as lactose or starch.

Buccal formulations that include a PAK activator include, but are not limited to, U.S. Pat. Nos. 4,229,447, 4,596,795, 4,755,386, and 5,739,136. In addition, the buccal dosage forms described herein optionally further include a bioerodible (hydrolysable) polymeric carrier that also serves to adhere the dosage form to the buccal mucosa. The buccal dosage form is fabricated so as to erode gradually over a predetermined time period, wherein the delivery of a PAK activator, is provided essentially throughout. Buccal drug delivery avoids the disadvantages encountered with oral drug administration, e.g., slow absorption, degradation of the active agent by fluids present in the gastrointestinal tract and/or first-pass inactivation in the liver. The bioerodible (hydrolysable) polymeric carrier generally comprises hydrophilic (water-soluble and water-swellable) polymers that adhere to the wet surface of the buccal mucosa. Examples of polymeric carriers useful herein include acrylic acid polymers and co, e.g., those known as “carbomers” (Carbopol®, which may be obtained from B.F. Goodrich, is one such polymer). Other components also be incorporated into the buccal dosage forms described herein include, but are not limited to, disintegrants, diluents, binders, lubricants, flavoring, colorants, preservatives, and the like. For buccal or sublingual administration, the compositions optionally take the form of tablets, lozenges, or gels formulated in a conventional manner.

Transdermal formulations of a PAK activator are administered for example by those described in U.S. Pat. Nos. 3,598,122, 3,598,123, 3,710,795, 3,731,683, 3,742,951, 3,814,097, 3,921,636, 3,972,995, 3,993,072, 3,993,073, 3,996,934, 4,031,894, 4,060,084, 4,069,307, 4,077,407, 4,201,211, 4,230,105, 4,292,299, 4,292,303, 5,336,168, 5,665,378, 5,837,280, 5,869,090, 6,923,983, 6,929,801 and 6,946,144.

The transdermal formulations described herein include at least three components: (1) a formulation of a PAK activator; (2) a penetration enhancer; and (3) an aqueous adjuvant. In addition, transdermal formulations include components such as, but not limited to, gelling agents, creams and ointment bases, and the like. In some embodiments, the transdermal formulation further includes a woven or non-woven backing material to enhance absorption and prevent the removal of the transdermal formulation from the skin. In other embodiments, the transdermal formulations described herein maintain a saturated or supersaturated state to promote diffusion into the skin.

In some embodiments, formulations suitable for transdermal administration of a PAK activator employ transdermal delivery devices and transdermal delivery patches and are lipophilic emulsions or buffered, aqueous solutions, dissolved and/or dispersed in a polymer or an adhesive. Such patches are optionally constructed for continuous, pulsatile, or on demand delivery of pharmaceutical agents. Still further, transdermal delivery of a PAK activator is optionally accomplished by means of iontophoretic patches and the like. Additionally, transdermal patches provide controlled delivery of a PAK activator. The rate of absorption is optionally slowed by using rate-controlling membranes or by trapping a PAK activator within a polymer matrix or gel. Conversely, absorption enhancers are used to increase absorption. An absorption enhancer or carrier includes absorbable pharmaceutically acceptable solvents to assist passage through the skin. For example, transdermal devices are in the form of a bandage comprising a backing member, a reservoir containing a PAK activator optionally with carriers, optionally a rate controlling barrier to deliver a PAK activator to the skin of the host at a controlled and predetermined rate over a prolonged period of time, and means to secure the device to the skin.

Formulations that include a PAK activator suitable for intramuscular, subcutaneous, or intravenous injection include physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles including water, ethanol, polyols (propyleneglycol, polyethylene-glycol, glycerol, cremophor and the like), suitable mixtures thereof, vegetable oils (such as olive oil) and injectable organic esters such as ethyl oleate. Proper fluidity is maintained, for example, by the use of a coating such as lecithin, by the maintenance of the required particle size in the case of dispersions, and by the use of surfactants. Formulations suitable for subcutaneous injection also contain optional additives such as preserving, wetting, emulsifying, and dispensing agents.

For intravenous injections, a PAK activator is optionally formulated in aqueous solutions, preferably in physiologically compatible buffers such as Hank's solution, Ringer's solution, or physiological saline buffer. For transmucosal administration, penetrants appropriate to the barrier to be permeated are used in the formulation. For other parenteral injections, appropriate formulations include aqueous or nonaqueous solutions, preferably with physiologically compatible buffers or excipients.

Parenteral injections optionally involve bolus injection or continuous infusion. Formulations for injection are optionally presented in unit dosage form, e.g., in ampoules or in multi dose containers, with an added preservative. In some embodiments, the pharmaceutical composition described herein are in a form suitable for parenteral injection as a sterile suspensions, solutions or emulsions in oily or aqueous vehicles, and contain formulatory agents such as suspending, stabilizing and/or dispersing agents. Pharmaceutical formulations for parenteral administration include aqueous solutions of a PAK activator in water soluble form. Additionally, suspensions of a PAK activator are optionally prepared as appropriate oily injection suspensions.

In some embodiments, a PAK activator is administered topically and formulated into a variety of topically administrable compositions, such as solutions, suspensions, lotions, gels, pastes, medicated sticks, balms, creams or ointments. Such pharmaceutical compositions optionally contain solubilizers, stabilizers, tonicity enhancing agents, buffers and preservatives.

A PAK activator is also optionally formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, PEG, and the like. In suppository forms of the compositions, a low-melting wax such as, but not limited to, a mixture of fatty acid glycerides, optionally in combination with cocoa butter is first melted.

A PAK activator is optionally formulated for delivery across the blood-brain barrier. In some embodiments, provided herein is a pharmaceutical composition comprising a PAK activator and an agent that facilitates the transport of the PAK activator across the blood brain barrier. In certain embodiments, an agent that facilitates the transport of the PAK activator is covalently attached to a PAK activator. In some instances, PAK activators described herein are modified by covalent attachment to a lipophilic carrier or co-formulation with a lipophilic carrier. In some embodiments, a PAK activator is covalently attached to a lipophilic carrier, such as e.g., DHA, or a fatty acid. In some embodiments, a PAK activator is covalently attached to artificial low density lipoprotein particles. In some instances, carrier systems facilitate the passage of PAK activators described herein across the blood-brain barrier and include but are not limited to, the use of a dihydropyridine pyridinium salt carrier redox system for delivery of drug species across the blood brain barrier. In some instances a PAK activator described herein is coupled to a lipophilic phosphonate derivative. In certain instances, PAK activators described herein are conjugated to PEG-oligomers/polymers or aprotinin derivatives and analogs. In some instances, an increase in influx of a PAK activator described herein across the blood brain bather is achieved by modifying A PAK activator described herein (e.g., by reducing or increasing the number of charged groups on the compound) and enhancing affinity for a blood brain bather transporter. In certain instances, a PAK activator is co-administered with an agent that reduces or inhibits efflux across the blood brain barrier, e.g. an inhibitor of P-glycoprotein pump (PGP) mediated efflux (e.g., cyclosporin, SCH66336 (lonafarnib, Schering)).

### Examples of Methods of Dosing and Treatment Regimens

A PAK activator is optionally used in the preparation of medicaments for the prophylactic and/or therapeutic treatment of neuropsychiatric diseases or conditions that would benefit, at least in part, from amelioration. In addition, a method for treating any of the diseases or conditions described herein in a subject in need of such treatment, involves administration of pharmaceutical compositions containing at least one PAK activator described herein, or a pharmaceutically acceptable salt, pharmaceutically acceptable N-oxide, pharmaceutically active metabolite, pharmaceutically acceptable prodrug, or pharmaceutically acceptable solvate thereof, in therapeutically effective amounts to said subject.

In the case wherein the patient's condition does not improve, upon the doctor's discretion the administration of a PAK activator is optionally administered chronically, that is, for an extended period of time, including throughout the duration of the patient's life in order to ameliorate or otherwise control or limit the symptoms of the patient's disease or condition.

In the case wherein the patient's status does improve, upon the doctor's discretion the administration of a PAK activator is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced, as a function of the symptoms, to a level at which the improved disease, disorder or condition is retained. In some embodiments, patients require intermittent treatment on a long-term basis upon any recurrence of symptoms.

In some embodiments, the pharmaceutical composition described herein are in unit dosage forms suitable for single administration of precise dosages. In unit dosage form, the formulation is divided into unit doses containing appropriate quantities of one or more PAK activator. In some embodiments, the unit dosage is in the form of a package containing discrete quantities of the formulation. Non-limiting examples are packaged tablets or capsules, and powders in vials or ampoules. In some embodiments, aqueous suspension compositions are packaged in single-dose non-reclosable containers. Alternatively, multiple-dose reclosable containers are used, in which case it is typical to include a preservative in the composition. By way of example only, formulations for parenteral injection are presented in unit dosage form, which include, but are not limited to ampoules, or in multi dose containers, with an added preservative.

The daily dosages appropriate for a PAK activator are from about 0.01 to 2.5 mg/kg per body weight. An indicated daily dosage in the larger mammal, including, but not limited to, humans, is in the range from about 0.5 mg to about 100 mg, conveniently administered in divided doses, including, but not limited to, up to four times a day or in extended release form. Suitable unit dosage forms for oral administration include from about 1 to 50 mg active ingredient. The foregoing ranges are merely suggestive, as the number of variables in regard to an individual treatment regime is large, and considerable excursions from these recommended values are not uncommon. Such dosages are optionally altered depending on a number of variables, not limited to the activity of a PAK activator used, the disease or condition to be treated, the mode of administration, the requirements of the individual subject, the severity of the disease or condition being treated, and the judgment of the practitioner.

Toxicity and therapeutic efficacy of such therapeutic regimens are optionally determined in cell cultures or experimental animals, including, but not limited to, the determination of the LD50 (the dose lethal to 50% of the population) and the ED50 (the dose therapeutically effective in 50% of the population). The dose ratio between the toxic and therapeutic effects is the therapeutic index, which is expressed as the ratio between LD50 and ED50. PAK activators exhibiting high therapeutic indices are preferred. The data obtained from cell culture assays and animal studies is optionally used in formulating a range of dosage for use in human. The dosage of such PAK activators lies preferably within a range of circulating concentrations that include the ED50 with minimal toxicity. The dosage optionally varies within this range depending upon the dosage form employed and the route of administration utilized. Optionally, the dosage is based on a determination of the amount a PAK activator that is able to cross the blood-brain barrier, e.g., by an assay described by Gomes and Seoares-da-Silvia in Brain Res. 829:143-150 (1999).

### Exemplary Subjects for PAK Activator Administration

PAK activator compositions described herein are optionally administered to a subject that already suffers from or is at risk of suffering from neurological and/or neuropsychiatric diseases and has been prescribed compounds directed toward that disease. In some embodiments, a PAK activator is administered to a subject suffering from or at risk of suffering from a psychotic disorder (e.g., schizophrenia) and has been prescribed therapeutic agents/treatments for treating psychotic disorders that include, but are not limited to, any of the following: typical antipsychotics, e.g., Chlorpromazine (Largactil, Thorazine), Fluphenazine (Prolixin), Haloperidol (Haldol, Serenace), Molindone, Thiothixene (Navane), Thioridazine (Mellaril), Trifluoperazine (Stelazine), Loxapine, Perphenazine, Prochlorperazine (Compazine, Buccastem, Stemetil), Pimozide (Orap), Zuclopenthixol; and atypical antipsychotics, e.g., LY2140023, Clozapine, Risperidone, Olanzapine, Quetiapine, Ziprasidone, Aripiprazole, Paliperidone, Asenapine, Iloperidone, Sertindole, Zotepine, Amisulpride, Bifeprunox, and Melperone.

In some embodiments, a PAK activator is administered to a subject suffering from or at risk of suffering from a mood disorder (e.g., clinical depression) and has been prescribed therapeutic agents/treatments for treating mood disorders that include, but are not limited to, any of the following: selective serotonin reuptake inhibitors (SSRIs) such as citalopram (Celexa), escitalopram (Lexapro, Esipram), fluoxetine (Prozac), paroxetine (Paxil, Seroxat), sertraline (Zoloft), fluvoxamine (Luvox); serotonin-norepinephrine reuptake inhibitors (SNRIs) such as venlafaxine (Effexor), desvenlafaxine, nefazodone, milnacipran, duloxetinc (Cymbalta), bicifadine; tricyclic antidepressants such as amitriptyline, amoxapine, butriptyline, clomipramine, desipramine, dosulepin, doxepin, impramine, lofepramine, nortriptyline; monoamine oxidase inhibitors (MAOIs) such as isocarboxazid, linezolid, moclobemide, nialamide, phenelzine, selegiline, tranylcypromine, trimipramine; and other agents such as mirtazapine, reboxetine, viloxazine, malprotiline, and bupropion.

In some embodiments, a PAK activator is administered to a subject suffering from or at risk of suffering from epilepsy and has been prescribed therapeutic agents/treatments for treating epilepsy that include, but are not limited to, any of the following: carbamazepine, clobazam, clonazepam, clorazepate, ethosuximide, felbamate, fosphenytoin, gabapentin, lamotrigine, levetiracetam, oxcarbazepine, phenobarbital, phenyloin, pregabalin, primidone, sodium valproate, tiagabine, topiramate, valproate semisodium, valproic acid, vigabatrin, and zonisamide.

In some embodiments, a PAK activator is administered to a subject suffering from or at risk of suffering from or at risk of suffering from Huntington's disease and is prescribed therapeutic agents/treatments for treating Huntington's disease that include, but are not limited to, any of the following: omega-3 fatty acids, miraxion, dopamine receptor blockers, creatine, Coenzyme Q10, minocycline, antioxidants, antidepressants (notably, but not exclusively, selective serotonin reuptake inhibitors SSRIs, such as sertraline, fluoxetine, and paroxetine), select dopamine antagonists, such as tetrabenazine; and RNAi knockdown of mutant huntingtin (mHtt).

In some embodiments, a PAK activator is administered to a subject suffering from or at risk of suffering from Parkinson's Disease and is prescribed therapeutic agents/treatments for treating Parkinson's Disease that include, but are not limited to any of the following: L-dopa, carbidopa, benserazide, tolcapone, entacapone, bromocriptine, pergolide, pramipexole, ropinirole, cabergoline, apomorphine, lisuride, selegiline, or rasagiline.

### Combination Treatments

PAK activator compositions described herein are also optionally used in combination with other therapeutic reagents that are selected for their therapeutic value for the condition to be treated. In general, the compositions described herein and, in embodiments where combinational therapy is employed, other agents do not have to be administered in the same pharmaceutical composition, and, because of different physical and chemical characteristics, are optionally administered by different routes. The initial administration is generally made according to established protocols, and then, based upon the observed effects, the dosage, modes of administration and times of administration subsequently modified.

In certain instances, it is appropriate to administer at least one PAK activator composition described herein in combination with another therapeutic agent. By way of example only, if one of the side effects experienced by a patient upon receiving one of a PAK activator compositions described herein is nausea, then it is appropriate to administer an anti-nausea agent in combination with the initial therapeutic agent. Or, by way of example only, the therapeutic effectiveness of a PAK activator is enhanced by administration of an adjuvant (i.e., by itself the adjuvant has minimal therapeutic benefit, but in combination with another therapeutic agent, the overall therapeutic benefit to the patient is enhanced). Or, by way of example only, the benefit experienced by a patient is increased by administering a PAK activator with another therapeutic agent (which also includes a therapeutic regimen) that also has therapeutic benefit. In any case, regardless of the disease, disorder or condition being treated, the overall benefit experienced by the patient is either simply additive of the two therapeutic agents or the patient experiences a synergistic benefit.

Therapeutically-effective dosages vary when the drugs are used in treatment combinations. Methods for experimentally determining therapeutically-effective dosages of drugs and other agents for use in combination treatment regimens are documented methodologies. One example of such a method is the use of metronomic dosing, i.e., providing more frequent, lower doses in order to minimize toxic side effects. Combination treatment further includes periodic treatments that start and stop at various times to assist with the clinical management of the patient.

In any case, the multiple therapeutic agents (one of which is a PAK activator described herein) is administered in any order, or even simultaneously. If simultaneously, the multiple therapeutic agents are optionally provided in a single, unified form, or in multiple forms (by way of example only, either as a single pill or as two separate pills). In some embodiments, one of the therapeutic agents is given in multiple doses, or both are given as multiple doses. If not simultaneous, the timing between the multiple doses optionally varies from more than zero weeks to less than four weeks. In addition, the combination methods, compositions and formulations are not to be limited to the use of only two agents; the use of multiple therapeutic combinations are also envisioned.

It is understood that the dosage regimen to treat, prevent, or ameliorate the condition(s) for which relief is sought, is optionally modified in accordance with a variety of factors. These factors include the disorder from which the subject suffers, as well as the age, weight, sex, diet, and medical condition of the subject. Thus, the dosage regimen actually employed varies widely, in some embodiments, and therefore deviates from the dosage regimens set forth herein.

The pharmaceutical agents which make up the combination therapy disclosed herein are optionally a combined dosage form or in separate dosage forms intended for substantially simultaneous administration. The pharmaceutical agents that make up the combination therapy are optionally also be administered sequentially, with either therapeutic compound being administered by a regimen calling for two-step administration. The two-step administration regimen optionally calls for sequential administration of the active agents or spaced-apart administration of the separate active agents. The time period between the multiple administration steps ranges from, a few minutes to several hours, depending upon the properties of each pharmaceutical agent, such as potency, solubility, bioavailability, plasma half-life and kinetic profile of the pharmaceutical agent. Circadian variation of the target molecule concentration are optionally used to determine the optimal dose interval.

In addition, a PAK activator is optionally used in combination with procedures that provide additional or synergistic benefit to the patient. By way of example only, patients are expected to find therapeutic and/or prophylactic benefit in the methods described herein, wherein pharmaceutical composition of a PAK activator and/or combinations with other therapeutics are combined with genetic testing to determine whether that individual is a carrier of a mutant gene that is correlated with certain diseases or conditions.

A PAK activator and the additional therapy(ies) are optionally administered before, during or after the occurrence of a disease or condition, and the timing of administering the composition containing a PAK activator varies in some embodiments. Thus, for example, a PAK activator is used as a prophylactic and is administered continuously to subjects with a propensity to develop conditions or diseases in order to prevent the occurrence of the disease or condition. PAK activators and compositions are optionally administered to a subject during or as soon as possible after the onset of the symptoms. The administration of the compounds are optionally initiated within the first 48 hours of the onset of the symptoms, preferably within the first 48 hours of the onset of the symptoms, more preferably within the first 6 hours of the onset of the symptoms, and most preferably within 3 hours of the onset of the symptoms. The initial administration is optionally via any route practical, such as, for example, an intravenous injection, a bolus injection, infusion over 5 minutes to about 5 hours, a pill, a capsule, transdermal patch, buccal delivery, and the like, or combination thereof. A PAK activator is preferably administered as soon as is practicable after the onset of a disease or condition is detected or suspected, and for a length of time necessary for the treatment of the disease, such as, for example, from about 1 month to about 3 months. The length of treatment optionally varies for each subject, and the length is then determined using the known criteria. For example, a PAK activator or a formulation containing a PAK activator is administered for at least 2 weeks, preferably about 1 month to about 5 years, and more preferably from about 1 month to about 3 years.

Exemplary Therapeutic Agents for Use in Combination with a PAK Activator Composition

### Agents for Treating Psychotic Disorders

Where a subject is suffering from or at risk of suffering from a psychotic disorder (e.g., schizophrenia), a PAK activator composition described herein is optionally used together with one or more agents or methods for treating a psychotic disorder in any combination. Examples of therapeutic agents/treatments for treating a psychotic disorder include, but are not limited to, any of the following: typical antipsychotics, e.g., Chlorpromazine (Largactil, Thorazine), Fluphenazine (Prolixin), Haloperidol (Haldol, Serenace), Molindone, Thiothixene (Navane), Thioridazine (Mellaril), Trifluoperazine (Stelazine), Loxapine, Perphenazine, Prochlorperazine (Compazine, Buccastem, Stemetil), Pimozide (Orap), Zuclopenthixol; and atypical antipsychotics, e.g., LY2140023, Clozapine, Risperidone, Olanzapine, Quetiapine, Ziprasidone, Aripiprazole, Paliperidone, Asenapine, Iloperidone, Sertindole, Zotepine, Amisulpride, Bifeprunox, and Melperone.

### Agents for Treating Mood Disorders

Where a subject is suffering from or at risk of suffering from a mood disorder (e.g., clinical depression), a PAK activator composition described herein is optionally used together with one or more agents or methods for treating a mood disorder in any combination. Examples of therapeutic agents/treatments for treating a mood disorder include, but are not limited to, any of the following: selective serotonin reuptake inhibitors (SSRIs) such as citalopram (Celexa), escitalopram (Lexapro, Esipram), fluoxetine (Prozac), paroxetine (Paxil, Seroxat), sertraline (Zoloft), fluvoxamine (Luvox); serotonin-norepinephrine reuptake inhibitors (SNRIs) such as venlafaxine (Effexor), desvenlafaxine, nefazodone, milnacipran, duloxetine (Cymbalta), bicifadine; tricyclic antidepressants such as amitriptyline, amoxapine, butriptyline, clomipramine, desipramine, dosulepin, doxepin, impramine, lofepramine, nortriptyline; monoamine oxidase inhibitors (MAOIs) such as isocarboxazid, linezolid, moclobemide, nialamide, phenelzine, selegiline, tranylcypromine, trimipramine; and other agents such as mirtazapine, reboxetine, viloxazine, malprotiline, and bupropion.

### Agents for Treating Epilepsy

Where a subject is suffering from or at risk of suffering from epilepsy, a PAK activator composition described herein is optionally used together with one or more agents or methods for treating epilepsy in any combination. Examples of therapeutic agents/treatments for treating epilepsy include, but are not limited to, any of the following: carbamazepine, clobazam, clonazepam, clorazepate, ethosuximide, felbamate, fosphenytoin, gabapentin, lamotrigine, levetiracetam, oxcarbazepine, phenobarbital, phenyloin, pregabalin, primidone, sodium valproate, tiagabine, topiramate, valproate semisodium, valproic acid, vigabatrin, and zonisamide.

### Agents for Treating Huntington's Disease

Where a subject is suffering from or at risk of suffering from Huntington's disease, a PAK activator composition described herein is optionally used together with one or more agents or methods for treating Huntington's disease in any combination. Examples of therapeutic agents/treatments for treating Huntington's disease include, but are not limited to, any of the following: omega-3 fatty acids, miraxion, dopamine receptor blockers, creatine, Coenzyme Q10, minocycline, antioxidants, antidepressants (notably, but not exclusively, selective serotonin reuptake inhibitors SSRIs, such as sertraline, fluoxetine, and paroxetine), select dopamine antagonists, such as tetrabenazine; and RNAi knockdown of mutant huntingtin (mHtt).

### Agents for Treating Parkinson's Disease

Where a subject is suffering from or at risk of suffering from Parkinson's Disease, a PAK activator composition described herein is optionally used together with one or more agents or methods for treating Parkinson's disease in any combination. Examples of therapeutic agents/treatments for treating Parkinson's Disease include, but are not limited to any of the following: L-dopa, carbidopa, benserazide, tolcapone, entacapone, bromocriptine, pergolide, pramipexole, ropinirole, cabergoline, apomorphine, lisuride, selegiline, or rasagiline.

### EXAMPLES

The following specific examples are to be construed as merely illustrative, and not limitative of the remainder of the disclosure in any way whatsoever.

### Example 1

### Treatment of Schizophrenia by Administration of a PAK Activator in an Animal Model

The ability of a PAK activator sphingosine to ameliorate behavioral and anatomical symptoms of schizophrenia (i.e., their mouse analogs) is tested in a dominant-negative DISC1 mouse model of schizophrenia (Hikida et al (2007), Proc Natl Acad Sci USA, 104(36):14501-14506).

Forty DISC1 mice (ages 5-8 months) on a C57BL6 strain background are divided into a Sphingosine treatment group (1 mg/kg i.p.) and a placebo group (0.1% DMSO in physiological saline solution) and analyzed for behavioral differences in open field, prepulse inhibition, and hidden food behavioral tests, with an interval of about one week between each type of test. In the open field test, each mouse is placed in a novel open field box (40 cm×40 cm; San Diego Instruments, San Diego, Calif.) for two hours. Horizontal and vertical locomotor activities in the periphery as well as the center area are automatically recorded by an infrared activity monitor (San Diego Instruments). Single breaks are reported as “counts.” In this behavioral test, a significant reduction in total activity in the Sphingosine group relative to the placebo group indicates a possible treatment effect.

In the hidden food test, mice are food-deprived for 24 h. After habituation to a new cage for 5 min, a food pellet is hidden under the cage bedding. The time it takes for the mouse to find the food pellet is measured until a maximum of 10 min is reached. In this behavioral test, a significant reduction in time to find the food pellet in the Sphingosine group relative to the placebo group is indicative of a successful treatment effect.

In the prepulse inhibition test, acoustic startle and prepulse inhibition responses are measured in a startle chamber (San Diego Instruments). Each mouse is subjected to six sets of seven trail types distributed pseudorandomly: pulse-alone trials, prepulse-pulse trials, and no-stimulus trials. The pulse used is 120 dB and the prepulse is 74 dB. A significant increase in the prepulse inhibition response in the Sphingosine group relative to the placebo group is indicative of a successful treatment effect.

In the forced swim test, each mouse is put in a large plastic cylinder, which is half-filled with room temperature water. The test duration is 6 min, during which the swim/immobility times are recorded. In this behavioral test, a significant reduction in immobility in the Sphingosine group relative to the placebo group is indicative of a successful treatment effect.

In order to evaluate the ability of Sphingosine to alter brain morphology, an MRI study is conducted on placebo-treated and sphingosine-treated groups of DISC 1-DN mice. In vivo MRI experiments are performed on an 11.7T Bruker Biospec small animal imaging system. A three-dimensional, fast-spin echo, diffusion weighted (DW) imaging sequence with twin navigation echoes is used to assess the ratio of lateral ventricle volume to total brain volume. A decrease in this ratio in the Sphingosine-treated group relative to the ratio observed in the placebo-group is indicative of a successful treatment effect.

Statistical Analysis. Statistical analysis is performed by ANOVA or repeated ANOVA. Differences between groups are considered significant at p<0.05.

### Example 2

### Treatment of Clinical Depression by Administration of a PAK Activator in an Animal Model

A rat olfactory bulbectomy (OBX) model of clinical depression (see, e.g., van Riezen et al (1990), Pharmacol Ther, 47(1):21-34) is used to evaluate treatment of clinical depression with an indirect PAK activator, a BDNF-human insulin antibody fusion protein (BDNF-HIRAb) agonist of the TrkB receptor, generated as described in U.S. patent application Ser. No. 11/245,546. Dendritic spine density and morphology are compared in treated and untreated groups of animals as described below. It is expected that treatment of OBX animals with BDNF-HIRAb will cause an increase in spine density relative to that observed in untreated OBX animals.

All experiments are performed in strict accordance with NIH standards for laboratory animal use. The study uses 48 adult male Sprague-Dawley rats (230-280 g) housed in groups of four animals (two sham and two OBX), as indicated in van Riezen et al supra, in a controlled environment with food and water available ad libitum. Half of the experimental animals (n=24) undergo bilateral olfactory bulbectomy (OBX) while the other half undergot sham surgery (n=24). Upon completion of surgery, animals are allowed to recover for 2 weeks prior to behavioral testing. This is necessary to: 1) allow for the recovery of animal body weight which is reduced following surgery, 2) allow complete healing of superficial surgical sites, and) “bulbectomy syndrome” develops during the first 2 weeks postsurgery.

Two weeks after surgery, OBX and sham-operated animals are subdivided into one of four experimental conditions. One group of OBX animals is administered daily injections of 0.9% saline (n=6 for each surgical condition) or BDNF-HIR-MAb fusion protein (5 mg/kg) (n=6 for each surgical condition). These groups are included to examine the effect of chronic administration of an indirect PAK activator on olfactory bulbectomized animals (2 weeks postsurgical recovery+2 weeks BDNF-HIRMAb treatment). Injections are given at the same time each day and in the home cage of each animal. Groups of OBX and sham-operated animals receive no treatment during this 2-week period and serve as unhandled controls. These groups are necessary to examine the persistence of observed effects of OBX on dendritic spine density (4 weeks postsurgery). Animals receiving postsurgery drug treatment are sacrificed 24 h after the last injection.

Animals are perfused transcardially with 4% formaldehyde (in 0.1 M sodium phosphate buffer, pH=7.4) under deep anesthesia with sodium pentobarbital (60 mg/kg) at the completion of experimental procedures. Following fixation, brains are removed and placed in 4% formaldehyde (freshly depolymerized from para-formaldehyde) overnight. Brains are then sectioned at 100 μm on a vibratome and prepared for Golgi impregnation using a protocol adapted from previously described methods (Izzo et al., 1987). In brief, tissue sections are postfixed in 1% OsO₄ for 30 min and then washed in 0.1 M phosphate buffer (3×15 min). Sections are free-floated in 3.5% K₂Cr₂O₇ solution for 90 min, mounted between two microscope slides in a “sandwich” assembly, and rapidly immersed in a 1% AgNO₃ solution. The following day, sections are rinsed in ddH₂O, dehydrated in 70% and 100% ethanol, cleared with Histoclear™, and mounted on microscope slides with DPX.

Dendritic spines are counted on 1250× camera lucida images that include all spines observable in each focal plane occupied by the dendrite. Cells are analyzed only if they are fully impregnated (CA1: primary apical dendrites extended into stratum lacunosum moleculare and basilar dendrites extended into stratum oriens; CA3: primary apical dendrites extended into stratum lacunosum moleculare and basilar dendrites extended into stratum oriens; dentate gyrus: secondary dendrites extended from primary dendrite within the molecular layer), intact, and occurring in regions of the section that are free of blood vessels, precipitate, and/or other imperfections. Dendritic spines are counted along the entire length of secondary oblique dendritic processes (50-100 μm) extending from the primary apical dendrite within stratum radiatum of area CA1 and CA3. In CA1 and CA3, secondary dendrites are defined as those branches projecting directly from the primary apical dendrite exclusive of tertiary daughter branches. In addition, spines are counted along the length of secondary dendrites of granule cells in the dentate gyms to determine if effects are limited to CA1 and CA3. In dentate gyms, secondary dendrites are analyzed in the glutamatergic entorhinal input zone in the outer two-thirds of the molecular layer. Approximately 20 dendritic segments (10 in each cerebral hemisphere; 50-100 μm in length) in each hippocampal subregion (CA1, CA3, and dentate gyms) are examined for each experimental animal. Treatment conditions are coded throughout the entire process of cell identification, spine counting, dendritic length analysis, and subsequent data analysis. Analysis of variance and Tukey post-hoc pairwise comparisons are used to assess differences between experimental groups.

When significant changes in dendritic spine density are observed, camera lucida images and the Zeiss CLSM measurement program are used to quantify the number and length of secondary dendrites. This analysis is necessary as apparent changes in dendritic spine density can result from an increase or decrease in the length of dendrites and not the formation or loss of spines per se. Photomicrographs are obtained with a helium-neon 633 laser and Zeiss 410 confocal laser scanning microscope.

### Example 3

### Treatment of Epilepsy by Administration of a PAK Activator in an Animal Model

A rat tetanus toxin model of epilepsy is used to evaluate treatment of epilepsy with an indirect PAK activator, an AAV constitutively active (T422E) PAK3 (CA-PAK3) expression vector. Details of AAV construction are described in, e.g., U.S. Pat. No. 7,244,423.

Wistar rat pups (Harlan Sprague Dawley, Indianapolis, Ind.), 10 d of age, are anesthetized with an intraperitoneal injection of ketamine and xylazine (33 and 1.5 mg/kg, respectively). When necessary, this is supplemented by inhalation of methoxyflurane (Metofane). Tetanus toxin solution to be injected is generated by dissolving 2.5 or 5 mg of tetanus toxin in 20 or 40 nl of sterile saline solution. Afterwards, the tetanus toxin solution is coinjected into the right hippocampus along with 10⁸ particles of AAV-CA-PAK3.

To inject tetanus toxin and the AAV-CA-PAK3, the pups are placed in an infant rat stereotaxic head holder, a midline incision is made, and a small hole is drilled in the skull. The stereotaxic coordinates for injection are: anteroposterior, −2.1 mm; mediolateral, 3.0 mm from the bregma; and dorsoventral, −2.95 mm from the dural surface. The toxin and AAV particles are slowly injected at 4 nl/min. After injection, the needle is left in place for 15 min to reduce reflux up the needle track. During injections, the body temperature of rat pups is maintained by a warmed (electrically regulated) metal plate. Littermates, stereotaxically injected with sterile saline, or untreated rats serve as controls.

The frequency of behavioral seizures is monitored for 1 hr/day for 10 consecutive days after tetanus toxin/AAV injections. The types and duration of seizures are scored. Wild running seizures are most easily identified.

After seizure scoring on the 10^{th} day animals are perfused transcardially and dendritic spines in the CA3 region are counted and analyzed as described in Example 2.

The t test for comparison of two independent means is used in comparing the number of seizures in treated vs untreated rats and in comparing dendritic and axon arbors in experimental and control rats. When data are not normally distributed, a Mann-Whitney U test is used. Sigma Stat is used to perform all statistical tests.

### Example 4

### In Vivo Monitoring of Dendritic Spine Plasticity in Double Transgenic GFP-M/DN-DISC1 Mice Treated with a PAK Activator

In the following experiment, dendritic spine plasticity is directly monitored in vivo by two photon laser scanning microscopy (TPLSM) in double transgenic GFP-M/DN-DISC1 mice treated with a PAK activator or a placebo. Mice (C57BL/6) expressing GFP in a subset of cortical layer 5 neurons (transgenic line GFP-M described in Feng et al., 2000, Neuron 28:41-51) are crossed with DN-DISC1 C57BL/6 DN-DISC1 mice (Hikida et al (2007), Proc Natl Acad Sci USA, 104(36):14501-14506) to obtain heterozygous transgenic mice, which are then crossed to obtain homozygous double transgenic GFPM/DN-DISC1 mice used in this study.

GFP-M/DN-DISC1 animals aged 28-61 days are anesthetized using avertin (16 body weight; Sigma, St. Louis, Mo.). The skull is exposed, scrubbed, and cleaned with ethanol. Primary visual, somatosensory, auditory, and motor cortices are identified based on stereotaxic coordinates, and their location is confirmed with tracer injections (see below).

Long-term imaging experiments are started at P40. The skull is thinned over the imaging area as described in Grutzendler et al, (2002), Nature, 420:812-816. A small metal bar is affixed to the skull. The metal bar is then screwed into a plate that connected directly to the microscope stage for stability during imaging. The metal bar also allows for maintaining head angle and position during different imaging sessions. At the end of the imaging session, animals are sutured and returned to their cage. Thirty animals previously imaged at P40 are then divided into a control group receiving 0.1% DMSO in physiological saline solution (i.p. once per day) and a treatment group administered sphingosine, a PAK activator, in 0.1% DMSO (1 mg/kg, i.p., once per day). During the subsequent imaging sessions (at P45, P50, P55, or P70), animals are reanesthetized and the skull is rethinned. The same imaging area is identified based on the blood vessel pattern and gross dendritic pattern, which generally remains stable over this time period.

At the end of the last imaging session, injections of cholera toxin subunit B coupled to Alexa Fluor 594 are made adjacent to imaged areas to facilitate identification of imaged cells and cortical areas after fixation. Mice are transcardially perfused and fixed with paraformaldehyde, and coronal sections are cut to verify the location of imaged cells. Sections are then mounted in buffer, coverslipped, and sealed. Images are collected using a Fluoview confocal microscope (Olympus Optical, Melville, N.Y.).

For in vivo two photon imaging, a two-photon laser scanning microscope is used as described in Majewska et al, (2000), Pflügers Arch, 441:398-408. The microscope consists of a modified Fluoview confocal scan head (Olympus Optical) and a titanium/sulphur laser providing 100 fs pulses at 80 MHz at a wavelength of 920 nm (Tsunami; Spectra-Physics, Menlo Park, Calif.) pumped by a 10 W solid-state source (Millenia; Spectra-Physics). Fluorescence is detected using photomultiplier tubes (HC125-02; Hamamatsu, Shizouka, Japan) in whole-field detection mode. The craniotomy over the visual cortex is initially identified under whole-field fluorescence illumination, and areas with superficial dendrites are identified using a 20×, 0.95 numerical aperture lens (IR2; Olympus Optical). Spiny dendrites are further identified under digital zoom (7-10×) using two-photon imaging, and spines 50-200 μm below the pial surface are studied. Image acquisition is accomplished using Fluoview software. For motility measurements, Z stacks taken 0.5-1 μm apart are acquired every 5 min for 2 h. For synapse turnover experiments, Z stacks of dendrites and axons are acquired at P40 and then again at P50 or P70. Dendrites and axons located in layers 1-3 are studied. Although both layer 5 and layer 6 neurons are labeled in the mice used in this study, only layer 5 neurons send a clear apical dendrite close to the pial surface thus, the data will come from spines on the apical tuft of layer 5 neurons and axons in superficial cortical layers.

Images are exported to Matlab (MathWorks, Natick, Mass.) in which they are processed using custom-written algorithms for image enhancement and alignment of the time series. For motility measurements (see Majewska et al, (2003), Proc Natl Acad Sci USA, 100:16024-16029) spines are analyzed on two-dimensional projections containing between 5 and 30 individual images; therefore, movements in the z dimension are not analyzed. Spine motility is defined as the average change in length per unit time (micrometers per minute). Lengths are measured from the base of the protrusion to its tip. The position of spines are compared on different imaging days. Spines that are farther than 0.5 μm laterally from their previous location are considered to be different spines. Values for stable spines are defined as the percentage of the original spine population present on the second day of imaging. Only areas that show high signal-to-noise ratio in all imaging sessions will be considered for analysis. Analysis is performed blind with respect to animal age and sensory cortical area. Spine motility (e.g., spine turnover), morphology, and density are then compared between control and treatment groups. It is expected that treatment with a PAK activator sphingosine will lead to reduced spine turnover or increased spine morphogenesis thereby leading to increased spine density in sphingosine-treated animals relative to untreated control animals.

### Example 5

### Clinical Trial: Treatment of Schizophrenia with a PAK Activator

The following human clinical trial is performed to determine the safety and efficacy of a PAK activator sphingosine for the treatment of schizophrenia.

Sixty patients are recruited via referrals from community mental health teams, after the patients have been diagnosed with schizophrenia using the Structured Clinical Interview for DSM-IV (“SCID”; First et al., (1995), Structured Clinical Interview for DSM-IV Axis I Disorders, Patient Edition (SCID-P), version 2, New York State Psychiatric Institute, Biometrics Research, New York)

A screening visit is arranged and a full explanation of the study prior to screening is provided if the patient appeared suitable for and interested in taking part. For inclusion, all patients are required to meet the following criteria: (i) aged between 18 and 60 years, (ii) receiving stable treatment with an atypical (Risperidone, Olanzapine, Quetiapine) antipsychotic and have stable psychotic symptoms (i.e. no change in medication/dose of current medication over last 6 weeks and unlikely to require change in antipsychotic medication), (iii) negative urine screening for illicit drugs and negative pregnancy test for female patients, (iv) cooperative, able to ingest oral medication and willing to undertake repeated cognitive testing, (v) able to provide written informed consent, (vi) reading ability of not more than 40 errors on the National Adult Reading (Nelson et al, (1991)), and (vii) between 1 and 2 standard deviations (S.D.) below expected performance on the basis of age and education level on the California Verbal Learning Test (Delis et al., 1987). In addition, the following criteria are used to define unsuitable patients: (i) concurrent DSM-IV diagnosis, (ii) current treatment with benzodiazepines or antidepressants, (iii) history of neurodegenerative disorder in first degree relative (e.g. AD, Parkinson's disease, Huntington's disease, multiple sclerosis), (iv) history of DSM-IV substance dependence in the last year or substance abuse within last month, (v) lifetime history of trauma resulting in loss of consciousness for 1 h or longer, (vi) participation in another investigational drug trial within 6 weeks prior to study entry, (vii) recent (within last 3 months) history of suicidal or violent acts, and (viii) current diagnosis of uncontrollable seizure disorder, active peptic ulceration, severe and unstable cardiovascular disease or/and acute severe unstable asthma. The study procedures are approved by an institutional ethics review board. All patients in the study must provide written informed consent.

After screening has identified suitable patients that have provided informed consent, patients are placed on a single-blind placebo for 1 week. After 1 week on placebo (baseline), all patients complete a comprehensive cognitive test battery and undergo clinical assessments, and then are randomized into a double-blind protocol so that, half of the sample received sphingosine capsules and the remaining half received placebo for the next 24 weeks. Cognitive and clinical assessments are carried out again at 12 weeks and 24 weeks.

Patients assigned to the Sphingosine group will receive 1.5 mg twice a day for the first 2 weeks, 3 mg twice a day over the next 2 weeks, 4.5 mg twice a day dose for the next 2 weeks and then 6 mg twice a day for the remaining period so at the time of 12 weeks cognitive assessments all patients are on the maximum dose. The placebo group will receive identical appearing capsules containing ascorbic acid (100 mg).

Symptoms are rated within 4 days of cognitive testing using the Positive and Negative Syndrome scale (PANSS) (Kay et al. (1987), Schizophr Res, 13:261-276) on all three occasions, Side effects are also assessed within 4 days of testing using the Abnormal Involuntary Movement Scale (AIMS) (Guy, (1976), ECCDEU Assessment Manual for Psychopharmacology (revised), DREW Publication No. (ADM) National Institutes of Mental Health, Rockville, Md., pages 76-338). Inter-rater reliability is carried out for PANSS at 6 monthly intervals by rating exemplar cases based on patient interviews on videotapes.

The cognitive battery includes measures of executive functioning, verbal skills, verbal and spatial working memory, attention and psychomotor speed. The battery is administered to all patients on all three occasions in the same fixed order. Patients are allowed to take breaks as needed in order to obtain maximal performance at all times. Tests are administered and scored by trained psychologists who are blind to patients' group affiliations and are not involved in patients' treatment plan in any way.

Patients are told that the aim of the study is to investigate the cognitive effects of Sphingosine. They are requested to abstain from alcohol for at least 24 h prior to their scheduled cognitive testing.

The patients in the Sphingosine and placebo groups are compared on demographic, clinical, and cognitive variables obtained at baseline using independent sample I-tests.

The effects of Sphingosine on positive symptoms, negative symptoms, general psychopathology score, total PANSS scores, and the scores on the AIMS are analyzed (separately) by 2 (Treatment: Sphingosine, placebo)×3 (Time: baseline, 12 weeks, 24 weeks) analysis of variance (ANOVA).

All cognitive variables are first examined for their distribution properties, i.e., to ensure normality. The cognitive effects of Sphingosine over time are then evaluated by Treatment×Time ANOVA, performed separately for each variable, with Time as a within-subjects factor and Treatment as a between-subjects factor, followed by post-hoc mean comparisons wherever appropriate. All cognitive effects are then re-evaluated using ANOVA performed separately on change scores computed for each variable (12 weeks data minus baseline data, 24 weeks data minus baseline data). Alpha level for testing significance of effects is p=0.05.

The disclosed embodiments are provided by way of example only. Numerous variations, changes, and substitutions are feasible. It should be understood that various alternatives to the embodiments of the methods and compositions described herein may be employed in practicing the invention. It is intended that the following claims define the scope of the invention and that methods and compositions within the scope of these claims and their equivalents be covered thereby.

1. A method for treating a subject suffering from a neuropsychiatric condition, comprising administering to the subject a pharmacological composition comprising a therapeutically effective amount of at least one activator of a p21-activated kinase, wherein the neuropsychiatric condition is associated with abnormal dendritic spine density, abnormal dendritic spine size, abnormal dendritic spine plasticity, abnormal dendritic spine morphology or abnormal dendritic spine motility.

2. The method of claim 1, wherein the neuropsychiatric condition is a psychotic, cognitive, or mood disorder.

3. The method of claim 1, wherein the neuropsychiatric condition is associated with abnormal spine density.

4. (canceled)

5. The method of claim 1, wherein the neuropsychiatric condition is schizophrenia, clinical depression, epilepsy, age-related cognitive decline, Huntington's disease, Down's syndrome, Niemann-Pick disease, spongiform encephalitis, Lafora disease, Maple syrup urine disease, maternal phenylketonuria, atypical phenylketonuria, or tuberous sclerosis.

6. (canceled)

7. The method of claim 5, further comprising administering to the subject a therapeutically effective amount of an antipsychotic drug.

8. The method of claim 1, wherein the neuropsychiatric condition is clinical depression.

9. The method of claim 5, further comprising administering to the subject a therapeutically effective amount of an antidepressant drug.

10. The method of claim 1, wherein the at least one activator is an indirect activator.

11. The method of claim 10, wherein the indirect activator is a TrkB receptor agonist.

12. (canceled)

13. (canceled)

14. The method of claim 11, wherein the TrkB receptor agonist is a blood-brain barrier-permeable form of BDNF.

15. The method of claim 1, wherein the activator is a direct activator of p21-activated kinase.

16. The method of claim 15, wherein the direct activator comprises a constitutively active form of p21 kinase, Rac or Cdc42.

17.-25. (canceled)

26. The method of claim 10, wherein the indirect activator of PAK is a CDK5 inhibitor.
